(19) United States
(12) Patent Application Publication
(10) Pub. No.: US 2006/0223781 A1
(43) Pub. Date: Oct. 5, 2006

(54) Process for induction of intramolecular migration of sulfates, phosphates, and other oxyanions

(75) Inventors: Steven Y.C. Guo, Alameda, CA (US); H. Edward Conrad, Champaign, IL (US)

Correspondence Address: Steven Guo, 7 Evans Court, Alameda, CA, 94502, US

(73) Assignee: Neoparin, Inc.

(21) Appl. No.: 11/397,132

(22) Filed: Apr. 3, 2006

Related U.S. Application Data
(60) Provisional application No. 60/668,391, filed on Apr. 4, 2005.

(51) Int. Cl. A61K 31/737; A61K 31/728; C08B 37/10; C08B 37/00; C12P 19/28; A61K 31/727

(52) U.S. Cl. 514/54; 514/56; 536/21; 536/53; 536/54; 435/85

(57) ABSTRACT

This present invention provides methods for structural modification of a molecule containing a hydroxyl group and an oxyanion amide or oxyanion ester group on adjacent or nearby atomic positions. The oxyanion, such as sulfate and phosphate, can be transferred to the hydroxyl group when the molecule is treated with a carbodiimide or various other oxyanion activating agents, resulting in selective oxyanion transfer to the hydroxyl group. Certain polysaccharides, and especially glycosaminoglycans, may be sulfated at a specific hydroxyl group when such hydroxyl group is present adjacent to or nearby a sulfate, phosphate, or other oxyanionic group in ester- or amide-linked forms.

This application claims the benefit of U.S. Provisional Application No. 60/668,391, filed on Apr. 4, 2005

### BACKGROUND OF THE INVENTION

Sulfates, phosphates, and other oxyanions found in nature, or formed by chemical synthesis, may occur in linkages to amines (e.g., sulfamides, phosphoamides and other oxyanion amides) or in linkages to hydroxyl groups (e.g., sulfate esters, phosphate esters and oxyanion esters). In polysaccharides these oxyanion amides or oxyanion esters are found in structures with hydroxyl groups on adjacent carbons or on other nearby carbons. In addition to heparin and heparan sulfate, these structural features are found in many polysaccharides, and oligosaccharides or in monosaccharides, either naturally occurring or formed by cleavage of the polysaccharides. Additionally, synthetic polysaccharides, oligosaccharides and monosaccharides contain these structural features. Other natural or synthetic chemicals contain the same structural features. Examples include, but not limited to, glucosamine 2-sulfate, glucosamine 6-sulfate, and myo-inositol monophosphate. Furthermore, natural or synthetic amino compounds with hydroxyl groups on adjacent carbons or on other nearby carbons can be selectively N-sulfated on the amines by the method described of Lloyd, A. G., et al., Biochem. Pharmacol., 20:637-648, to generate chemicals with the same structural features, i.e. sulfamides with hydroxyl groups on adjacent carbons or on other nearby carbons. Other oxyanion amides can be made based on existing methods in similar fashion.

Regioselective sulfation or phosphorylation of polyhydroxyl compounds such as carbohydrates has always been technically challenging in the field. Even for monosaccharides or oligosaccharides, regioselective sulfation often requires multiple steps. For example, as described by Langston et al, (Helv Chem Acta 77, 2341), to sulfate a hydroxyl group at a particular position, one would need to protect other hydroxyls than the targeted acceptor hydroxyl to avoid unintended sulfation. To achieve site specific sulfation, the hydroxyls are protected by acetals or ethers. The unprotected and target hydroxyl group would then be sulfated using various sulfating reagents. Then, additional reactions must be carried out to reverse the protection of the hydroxyl groups. Two general approaches have been used extensively to sulfate polysaccharides. These include (a) the use of amine conjugates of sulfur trioxide as the sulfating agent (Gilbert (1962) Chem. Rev. 62: 550-589; Nagasawa, et al. (1986) Carbohyd. Res. 158: 183-190; Casu, et al. (1994) Carbohyd. Res. 263: 271-284), and (b) the use of either sulfuric acid or chlorosulfonic acid as the sulfating agent (Naggi, et al. (1987) Biochem. Pharmacol. 36: 1895-1900). Both methods have limitations on specificity and selectivity, and especially the regioselectivity. An attempt was made (Uchiyama & Nagasawa (1991) JBC 266: 6750-6760) to selectively sulfate the 3-OH of glucosamine in heparin. However, the reaction used also caused sulfation on 3-OH groups of uronic acid, and the yield was low. Enzymes that offer certain regioselective sulfations have been found very useful in research. However, these enzymes require specific substrates and the expensive sulfate donor, 3′-phosphoadenosine-5′-phosphosulfate. Thus, it is not feasible to use these enzymes for large scale sulfation at present time.

### SUMMARY OF THE INVENTION

The present invention provides new methods for causing the intramolecular migration of N-linked and O-linked oxyanions to adjacent or nearby hydroxyl groups in structures containing (a) an amino group and one or more hydroxyl groups, or (b) two or more hydroxyl groups. In these methods, these structures, or their tertiary or quaternary amine salts, are dissolved in an aprotic solvent, e.g., DMF, and are treated with a carbodiimide and an acid catalyst, e.g., sulfuric or hydrochloric acid. For these reactions, pH, type of acid, temperature, time intervals and solvents are chosen to control the rate, extent, and acceptor positions to which the oxyanion is transferred.

### DESCRIPTION OF THE DRAWINGS

FIG. 1, “Reaction Scheme of Intramolecular Oxyanion Transfer” shows the general structures of the reactants and products, particularly the donor and acceptor structures of this transfer reaction.

FIG. 2, “Reaction Scheme of Intramolecular N→O Sulfate Transfer”, shows the general structures of the structures and products of the reactions and shows the use of carbodiimides and an acid catalyst in a reaction that results in the transfer of the sulfate.

FIG. 3A, “Intramolecular Transfer from 2-N-Sulfate to 3-Hydroxyl of N-sulfated Glucosamine Residues in Heparin”, shows a specific example of the reactions in which an N-sulfated glucosamine in heparin transfers its N-sulfate group to the C3 hydroxyl group adjacent to the amino group of the same glucosamine residue.

FIG. 3B, “N→O Sulfate Transfer with n=3 or n=5 in a Heparin Trisaccharide Unit”, shows an example of the transfer of an N-sulfate group in a heparin trisaccharide unit to hydroxyl groups on uronic acid units that are spaced 3 to 5 positions away from the donor N-sulfate group on the glucosamine, i.e., the n in Aₙ in FIG. 1 is either 3 or 5.

FIG. 4, “Active Pentasaccharide (top panel) and Inactive Pentasaccharide (bottom panel) in Heparin”, shows pentasaccharide sequences in heparin that possess anticoagulant activity (top) and that lack anticoagulant activity (bottom) but that can be converted to active sequences using the reactions described herein.

### DETAILED DESCRIPTION OF THE INVENTION

Before the present method is described, it is understood that this invention is not limited to the particular methodology, protocols, and reagents described as these may vary. It is also to be understood that the terminology used herein is for the purpose of describing particular embodiments only, and is not intended to limit the scope of the present invention which will be limited only by claims. It must be noted that as used herein, the singular forms “a”, “an”, and “the” include plural reference unless the context clearly dictates otherwise.

Unless defined otherwise, all technical and scientific terms used herein have the same meanings as commonly understood by one of ordinary skill in the art to which this invention belongs. Although any methods and materials similar or equivalent to those described herein can be used in the practice or testing of the present invention, the preferred methods, devices, and materials are now described. All publications mentioned herein are incorporated herein by reference for the purpose of describing and disclosing the methodologies. Nothing herein is to be construed as an admission that the invention is not entitled to antedate such disclosure by virtue of prior invention.

A. Definitions

The use of certain terms in this specification preferably includes reference to the products or techniques defined below in relation to those terms.

“Monosaccharide,” as used herein, refers to a polyhydroxy alcohol containing a potential aldehyde or a ketone group, i.e., a simple sugar. Monosaccharide includes reference to naturally occurring simple sugars with 4 to 8 carbons, as well as simple sugars which have been chemically modified. Modified monosaccharides include, but are not limited to, monosaccharides that have increased or decreased sulfation or that have modified carboxyl, amino or hydroxyl groups.

“Uronic Acid,” as used herein, refers to a monosaccharide in which the primary alcoholic carbon is replaced by a carboxyl group.

“Hexosamine”, as used herein, refers to a hexose (a 6-carbon monosaccharide) in which the hydroxyl group at C2 is replaced with an amino group.

“Amino Sugar”, as used herein, refers to a hexosamine

“Polysaccharide,” as used herein, refers to a linear or branched polymer of more than 10 monosaccharides that are linked by means of glycosidic linkages.

“Oligosaccharide units,” as used herein, refers to a linear or branched polymer of 2 or more monosaccharides that are linked together by means of glycosidic linkages.

“Polyanion,” as used herein, refers to a molecule that possesses a large number of negative charges. “Polyanionic carbohydrates,” as used herein, includes reference to carbohydrates that possess a large number of negative charges.

“Glycosaminoglycan,” as used herein, includes reference to a polysaccharide composed of repeating disaccharide units. The disaccharides always contain an amino sugar (i.e., glucosamine or galactosamine) and one other monosaccharide, which may be a uronic acid (i.e., glucuronic acid or iduronic acid) as in hyaluronic acid, heparin, heparan sulfate, chondroitin sulfate or dermatan sulfate—or galactose as in keratan sulfate. The glycosaminoglycan chain may be sulfated on either moiety of the repeating disaccharide.

“Heparinoids,” as used herein, refer to all structural variations of heparin and heparan sulfate. These are oligomers of at least one disaccharide or polymers of at least 18 different disaccharides, which represent their monomeric units. The disaccharides are made up of a hexuronic acid residue and a D-glucosamine residue which are linked to each other and to the other disaccharides by 1→0.4 linkages. The glucosamine unit may be either N-acetylated (GlcNAc) or N-sulfated (GlcNSO₃). The uronic acid may be either a .β-D-glucuronic acid or an .α-L-iduronic acid residue. O-Sulfate substituents are found at C2 of some of the uronic acid residues and at C6 of some of the glucosamine residues. A general feature of these structures is that blocks of uronic acid→glucosamine disaccharides that contain high degrees of sulfation are separated from other such blocks by blocks of unsulfated glucosamine →.N-acetylated glucosamine disaccharides. The relative length of the sulfated and unsulfated blocks differ for different heparinoids, with relatively few unsulfated disaccharides in heparins and many blocks of GlcA→GlcNAc disaccharides in heparan sulfates. Thus, in all heparinoids there are a number of unsubstituted hydroxyl groups on both the glucosamine and the uronic acid residues.

“Heparin” (or, interchangeably, “standard heparin” (SH) or “unmodified heparin,” as used herein, includes reference to heparinoids that are highly sulfated and that have relatively high IdoA/GlcA ratios (1 to 10) and GlcNSO₃/GlcNAc ratios (1 to 10). Generally, heparin has an average molecular weight ranging from about 6,000 Daltons to 40,000 Daltons with an average of about 12,000 Daltons, depending on the source of the heparin and the methods used to isolate it. Heparin inhibits blood coagulation (i.e., it is an anticoagulant).

“De-O-Sulfated Heparin (De-OS-Heparin)”, as used herein, includes reference to heparins that are derived by de-O-sulfation of heparin using a previously established method.

“Heparan Sulfate” (HS), as used herein, includes reference to heparinoids that are less highly sulfated and that have relatively low IdoA/GlcA ratios (0.5-1.5) and GlcNSO₃/GlcNAc ratios (0.5-1.5).

“De-acetylated, N-sulfated Heparan Sulfate (DAc-NS-HS)” as used herein, includes reference to heparan sulfates that are derived by N-deacetylation of the amino groups of glucosamine and then N-sulfation of these glucosamine residues at their free amino groups using previously established methodology.

“Dermatan Sulfate” (DS), as used herein, includes reference to a heterogeneous glycosaminoglycan mixture that contains disaccharide repeat units consisting of N-acetyl-D-galactosamine and D-glucuronic acids, as well as disaccharide repeat units consisting of N-acetyl-D-galactosamine and L-iduronic acid. The N-acetyl-D-galactosamine residues may be sulfated on the 4 and/or the 6 position. The uronic acids are present with variable degrees of sulfation.

“De-acetylated, N-sulfated Dermatan Sulfate (DAc-NS-DS)” as used herein, includes reference to heparinoids that are derived by N-deacetylation and then N-sulfation of amino groups of the galactosamine residues of dermatan sulfate using previously established methodology.

“Chondroitin sulfate” (CS), as used herein, includes reference to a heterogeneous glycosaminoglycan mixture that contains disaccharide repeat units consisting of N-acetyl-D-galactosamine and D-glucuronic acids. The N-acetyl-D-galactosamine residues may be sulfated on the 4 and/or the 6 position.

“De-acetylated, N-sulfated Chondroitin Sulfate (DAc-NS-DS)” as used herein, includes reference to chondroitin sulfates that are derived by N-deacetylation and then N-sulfation of the amino groups of galactosamine residues of chondroitin sulfate using a previously established methodology.

“Hyaluronic acid”, as used herein, includes reference to a heterogeneous glycosaminoglycan mixture that contains disaccharide repeat units consisting of N-acetyl-D-glucosamine and D-glucuronic acids.

“De-acetylated, N-sulfated Hyaluronic Acid (DAc-NS-Hya)” as used herein, includes reference to heparinoids that are derived by N-deacetylation and then N-sulfation of the amino groups of the glucosamine residues of hyaluronic acid using previously established methodology.

“Chitosan”, as used herein, includes reference to a polymer of β-1-4-linked D-glucosamine residues, all of which contain free amino groups.

“N-sulfated Chitosan,” as used herein, includes reference to N-sulfated chitosan, prepared by N-sulfation using a previously established methodology.

“K5 polysaccharide (K5)”, as used herein, includes reference to heparinoids with the repeating disaccharide of GlcA→GlcNAc. K5 may be isolated from bacterial or animal origin.

“N-sulfate K5 (K5NS)”, as used herein, includes reference to heparinoids with the repeating disaccharide of GlcA→GlcNS, prepared by De-N-acetylation and N-sulfation of the K5 polysaccharide using previously established methodology.

“Epimerized N-sulfate K5 (EK5NS)”, as used herein, includes reference to heparinoids with the repeating disaccharide of either GlcA→GlcNS or IdoA→GlcNS, prepared by De-N-acetylation, N-sulfation and enzymatic epimerization using previously established methodology.

B. Description of Invention and Preferred Embodiments

The invention describes a novel process and method for regioselective sulfation, phosphorylation or similar modifications of hydroxyl groups when oxyanion amides or oxyanion esters are available on adjacent or nearby positions.

Sulfates, phosphates, and other oxyanions found in nature, or formed by chemical synthesis, occur in linkages to amines (e.g., sulfamides, phosphoamides and oxyanion amides) or in linkages to hydroxyl groups (e.g., sulfate esters, phosphate esters and oxyanion esters). In polysaccharides, such as heparin and heparan sulfate, these oxyanion amides or oxyanion esters are often found in structures with hydroxyl groups on adjacent carbons or on other nearby carbons. These structural features are available in many polysaccharides, oligosaccharides or monosaccharides that are either natural or synthetic. In addition to polysaccharides, other natural or synthetic chemicals contain similar structural features. Such chemicals include, but not limited to, glucosamine 2-sulfate, glucosamine 6-sulfate, and myo-inositol monophosphate.

Such structural features may be readily observed when amines are available with hydroxyl groups on adjacent carbons or on other nearby carbons. In a method described by Lloyd, A. G., et al. (1971), Biochem. Pharmacol., 20:637-648, amines may be selectively N-sulfated to generate chemicals containing sulfamides and hydroxyl groups on adjacent carbons or on other nearby carbons.

When such structural features are available or obtained through chemical modification, this invention discloses a new method that can induce transfer or migration of the oxyanion to the adjacent or nearby hydroxyl group, thus achieving selective modification of the hydroxyl group, which otherwise may not be readily modified through other conventional methods. For example, in the case of polysaccharides, multiple hydroxyl groups occur in one polysaccharide chain and may be nonspecifically sulfated through conventional methods. However, regioselective sulfation of a particular hydroxyl group proves to be difficult. This invention discloses a method that can selectively sulfate a certain hydroxyl group if such hydroxyl group is on a carbon that is adjacent to or nearby a carbon with an N-sulfate group or an O-sulfate group.

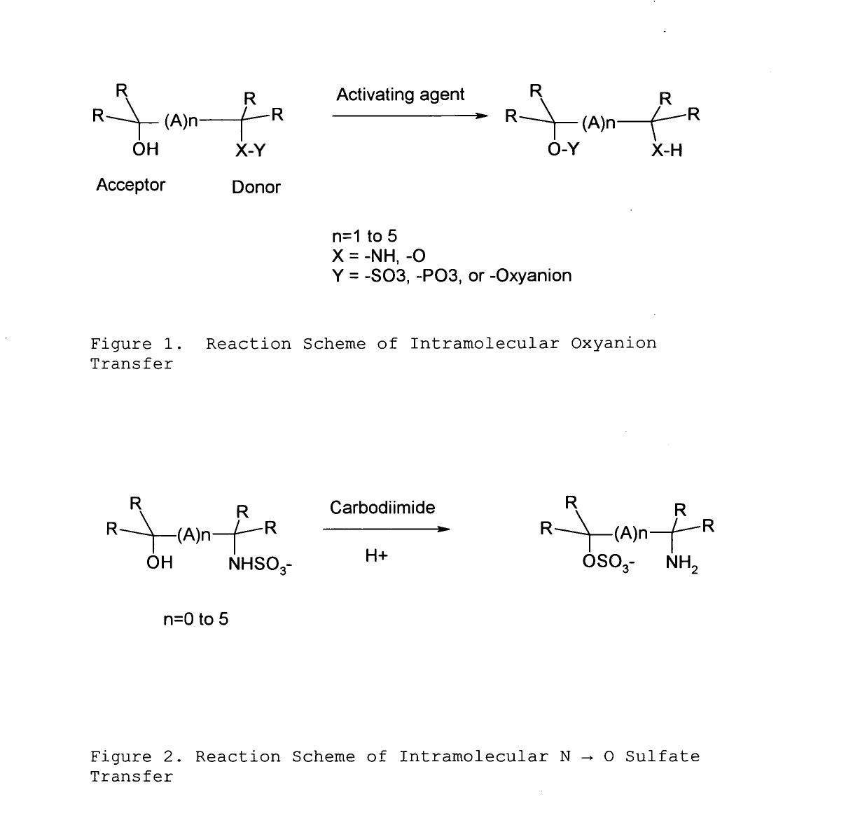

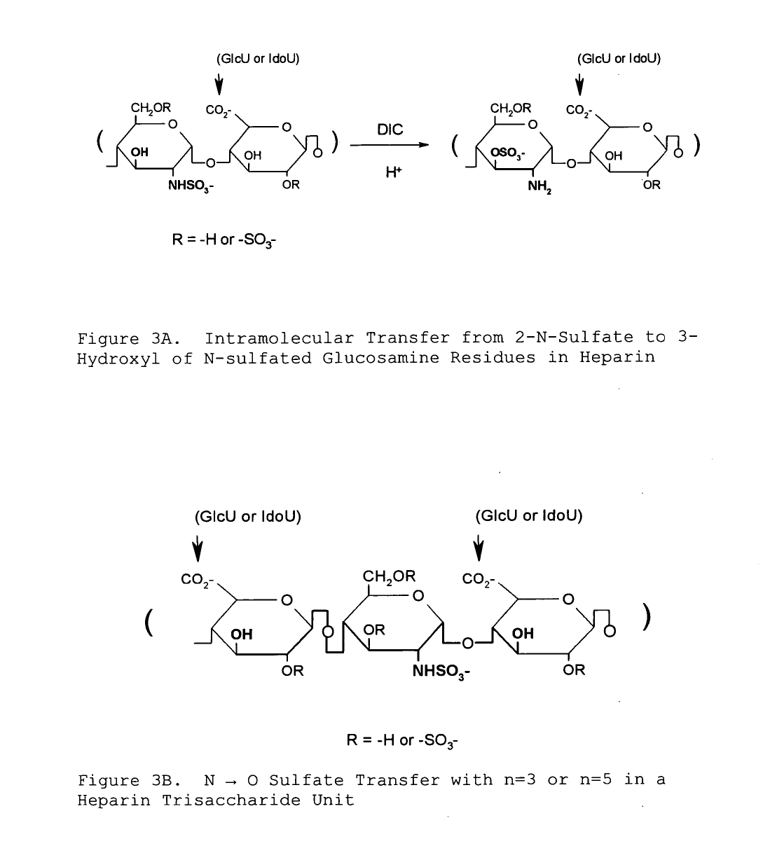

FIG. 1 illustrates the structural feature that serves as a substrate for the reaction described in the invention, Where, A refers to atoms that are commonly carbons in case of polysaccharides. However, it should be understood that in similar structures, A may represent other atoms, such as, but not limited to, oxygen, sulfur or silicon. In certain structural variations, A may be a combination of different atoms. For example, as seen in FIG. 3B the structural feature found in heparin disaccharide (depicted in bold) may contain an oxygen that links two carbons of different monosaccharides.

X includes, but is not limited to, NH or O, which is linked to an oxyanion group represented by the letter Y, including, but not limited to, SO₃H or PO₃H₂.

The oxyanion group in such a structure may be induced to transfer or migrate to the adjacent or nearby hydroxyl group when treated with activating agents, such as carbodiimides, or other activating agents that can generally activate the oxyanion into a reactive state in favor of detachment and migration to an adjacent or nearby hydroxyl group. The carbodiimide includes, but is not limited to, the following: dicyclohexylcarbodiimide (DCC); diisopropylcarbodiimide (DIC); 3-ethyl-[(3-dimethylaminopropyl)carbodiimide hydrochloride (EDC) and 1-cyclohexyl-3-(2-morpholoethyl)carbodiimide p-toluene sulfonate (CMC). Other activating agents include, but are not limited to, 1-hydroxybenzotriazole, N-hydroxysuccinimide, Benzotriazol-1-yloxytris(dimethylamine)phosphonium hexafluorophosphate (BOP), O-benzotriazol-1-yl-N,N,N′,N′-tetramethyluronium hexafluorophosphate (HBTU), Bis(2-oxo-3-oxazolidinyl)phosphinic chloride (BOP-Cl), (1H-1,2,3-benzotriazol-1-yloxy)tris(pyrrolidino)phosphonium hexafluorophosphate (Py-BOP), carbonyldiimidazole, 2-chloro-1-methylpyridinium Iodide, or any combination of the above activating agents.

FIG. 2 depicts a specific carbodiimide-induced reaction that would result in regioselective sulfation. As illustrated, a chemical with a structural feature containing a sulfamate and a hydroxyl group on an adjacent or nearby carbon may react to produce a new chemical with the amino group losing its sulfate and the hydroxyl group being sulfated. The regioselective O-sulfation is achieved by carbodiimide-induced sulfate transfer from the amino group to an adjacent or nearby hydroxyl group. This reaction will occur with a variety of compounds having the necessary structural features, including but not limited to, naturally occurring or synthetic hydroxyl-containing N-sulfated amines, N-sulfated amino sugar-containing structures such as monosaccharides, oligosaccharides and polysaccharides, including O-sulfated and/or oversulfated polysaccharides such as glycosaminoglycans. Such N-sulfated-amino sugar-containing oligosaccharides and polysaccharides are first converted into aprotic solvent-soluble salts that are then dissolved in an aprotic solvent. Carbodiimide is added to induce the N-sulfate transfer to an adjacent or near-by hydroxyl of these N-sulfated-amino sugar-containing oligosaccharides and polysaccharides under acidic conditions obtained by the addition of an acid such as sulfuric acid, hydrochloride acid, or phosphoric acid. When desirable, reaction conditions such as the type and concentration of acid, temperature, time intervals, substrate concentration and solvent are chosen to control the rate, extent, and acceptor positions to which the sulfate is transferred. When sulfuric acid or phosphoric acid is used, the acid concentration may be selected such that the acid serves not only as catalyst but also as a sulfating or phosphorylating agent. After N-sulfate is transferred to adjacent or nearby hydroxyl, the resulting free amine may be further N-sulfated or substituted by other groups according to previously established methods.

In FIG. 2, A refers to any atom. C—NH—SO₃— refers to a sulfate donor (see FIG. 1). C—OH refers to a sulfate acceptor. As shown in FIG. 1, similar structures with phosphates or other oxyanions may replace the sulfate groups.

For structures with n=0, N-sulfate transfer to the adjacent hydroxyl can be achieved in high yield with regioselectivity. For example, as depicted (in bold) in FIG. 3A, regioselective 3-O-sulfation of N-sulfated glucosamine residues in heparin is achieved almost stoichiometrically by transferring N-sulfate to the adjacent 3-hydroxyl.

For n=1 to n=5, conditions such as temperature, solvent type, reaction time, pH and reactant concentration may be selected to achieve the selective sulfation.

Depending the type of atoms (A) between donor and acceptor groups, a three dimensional configuration may structurally favor approximate contacts between donor and acceptor groups for the selective sulfation.

In a structure with n=3 or n=5, such as seen in FIG. 3B, the carbodiimide can induce N-sulfate transfer to 3-hydroxyl positions on uronic acids of either side of N-sulfated glucosamine residue.

It should be appreciated by those skilled in the art that the above processes shown in FIG. 2, FIG. 3A and FIG. 3B are applicable when the sulfate group is replaced with phosphate or other oxyanions. The process described by this patent provides an attractive alternative to the difficult multiple step processes for direct chemical introduction of the oxyanion into the desired acceptor position.

The method can be used in more complicated synthesis to achieve specific selectivity. When there is more than one hydroxyl group present in the substrate structure described herein, one may use this method to selectively modify the hydroxyl groups in sequential reactions. Selective sulfation may be further achieved when used in combination with phosphorylation and dephosphorylation. For instance, when 4-O-sulfated glucosamine is to be produced, one would start with N-phosphorylated-glucosamine by transferring N-phosphate to 3-O-phosphate. After 3-O-phosphorylated glucosamine is N-sulfated, then N-sulfate can be further transferred to 4-O-position to afford 3-O-phosphate-4-O-sulfate glucosamine. Because O-phosphate is more acid-labile than O-sulfate, 4-O-sulfated glucosamine can be obtained by limited acid hydrolysis using a prior method.

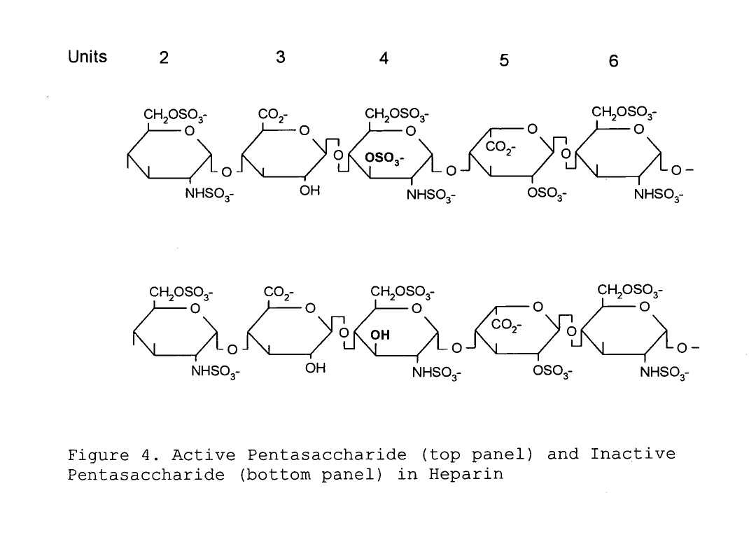

A case in which such a regioselective transfer reaction would be desirable would involve heparin or heparan sulfate, two structurally related polysaccharides which will inhibit the coagulation of blood. In heparin which has been studied extensively as an anticoagulant, the pentasaccharide sequence which is necessary for the anticoagulant activity is shown in the top panel of FIG. 4.

The sequence contains a critical glucosamine residue at unit 4 which is substituted with a 3-O-sulfate group. This pentasaccharide sequence is found on approximately one third of the polymeric chains in the most active heparin preparations. However, many of the glucosamine residues in heparin are N-sulfated, including some in sequences identical to this sequence, but lacking the 3-O-sulfate on unit 4, as shown as the bottom panel of FIG. 4. There are no direct and specific ways to regioselectively add sulfate groups to the 3-0 positions of the latter sequences to increase the anticoagulant activity of these heparin preparations. However, a procedure that induces the transfer of the N-sulfate on unit 4 to the 3-O-hydroxyl group would introduce the desired 3-O-sulfate group. Re—N-sulfation of the amino groups following such transfers is necessary to optimize the activity of these new sequences, thus completing the introduction of additional active sequences into the heparin preparation. Similarly, heparan sulfate contains a small number of the same active pentasaccharide sequences necessary for anticoagulant activity and its activity might also be increased following the sulfate migration and re-N-sulfation. This patent describes reaction conditions that cause transfer of N-sulfate groups of N-sulfated glucosamine residues in heparin and heparan sulfate to the C3 positions of these glucosamines in high yield.

Although among polysaccharides, heparin and heparan sulfate are the only natural structures containing N-sulfated amino sugars, there are many naturally occurring polysaccharides which contain N-acetylated amino sugars. These include the N-acetylglucosamine-containing polymers, hyaluronic acid, keratan sulfate, chitin, acharan sulfate, E. coli K5 polysaccharide, and their derivatives, as well as the N-acetylgalactosamine-containing polysaccharides, chondroitin sulfate, dermatan sulfate, E. coli K4 polysaccharide and their derivatives. Furthermore, many oligosaccharides and polysaccharides in glycoproteins, glycolipids and gangliosides contain free or N-acetyl-amino sugars. There are well-established methods for partial or complete removal of the acetyl groups from these amino sugar residues in these structures and for selectively N-sulfating or N-phosphorylating the resulting free amino groups. Such structurally modified structures thus become substrates for the N-sulfate transfer reactions, the N-phosphate transfer reactions and the oxyanion transfer reactions described in the present invention. Similarly, these and other polysaccharides, oligosaccharides, and monosaccharides can be partially or completely O-sulfated or O-phosphorylated on their hydroxyl groups to generate sulfate or phosphate esters. In these reactions, the monosaccharide residues can be selectively O-sulfated on their primary hydroxyl groups, for instance, the 6-O-hydroxyls of hexose residues of polysaccharides, oligosaccharides and monosaccharides. Then, the 6-O-sulfated saccharides can be further subjected to the sulfate transfer reaction under conditions that cause regioselective sulfation on the hydroxyl at C4, or C3, or C2 when optimized conditions are used. Similarly, reaction conditions may be worked out for transferring the 6-O-phosphate on phosphomannan to nearby hydroxyls. The further extension of transferring O-sulfate and O-phosphate to adjacent or nearby hydroxyl groups can be applied to all polyhydroxyl containing molecules. It is conceivable that oxyanions such as sulfate or phosphate may transfer from amino groups to adjacent or nearby hydroxyls, or from hydroxyls to adjacent or nearby hydroxyls in polysaccharides, oligosaccharides, monosaccharides, or any molecules containing the structural features described in FIG. 2, to generate a variety of new molecules. Consequently, this invention may convert biologically inactive molecules to biologically active species, or may enhance their biological activities, or may reduce side effects of their functions. More particularly, because the transfer is specific and selective, the invention may afford molecules that are not readily synthesized by current methods.

The present invention is further illustrated through the preparation of regioselective O-sulfation by carbodiimide-induced sulfate transfer from N-sulfated hexosamines to adjacent or near-by hydroxyl groups in natural or synthetic N-sulfated hexosamine and N-sulfated hexosamine-containing oligosaccharides and polysaccharides, including regioselective O-sulfation and/or oversulfated glycosaminoglycans. The methods generally comprise conversion of the N-sulfated hexosamine-containing oligosaccharides and polysaccharides into aprotic solvent-soluble salts, N-sulfate transfer to O-sulfate and/or O-sulfation of the N-sulfated hexosamine-containing oligosaccharides and polysaccharides with carbodiimide, and a mineral acid such as sulfuric acid, hydrochloride acid, phosphoric acid, and re-N-sulfation of the N→O transferred and/or O-sulfated product. The specific conditions employed during the above-described process enable one to obtain a regioselective sulfation of the N-sulfated hexosamine-containing oligosaccharides and polysaccharides, i.e., the O-sulfation at the selective position not being achievable by conventional method.

In the first step of the process, an alkali metal salt, e.g., sodium, salt of N-sulfated hexosamine-containing polysaccharide is converted to an amine salt, such as pyridine or tributylamine salt, or to a long chain quatenary amine salt. Examples of suitable amine salts include, but are not limited to, the following: trimethylamine, triethylamine, tripropylamine, tributylamine and quaternary ammonium salts. In a preferred embodiment, the amine salt is a tertiary amine salt such as pyridinium or a tributylamine salt. In another preferred embodiment, the amine salt is a quaternary ammonium salt, such as cetylpyridium, benzethonium or cetyltrimethylammonium salts. These amine salts can be prepared by various ion exchange methods or by precipitation approaches.

The N-sulfated hexosamine-containing polysaccharide can be converted to its amine salt using standard methods and procedures known to and used by those of skill in the art. For instance, the tertiary amine salt can be obtained by ion exchange chromatography or, alternatively, by batch ion-exchange. More particularly, to generate the tertiary amine salt of the N-sulfated hexosamine-containing-containing polysaccharide by ion exchange chromatography, the N-sulfated hexosamine-containing polysaccharide is dissolved in double distilled water, cooled to 2° C. to 8° C., and loaded onto an ion exchange column at refrigerated temperature (2-8° C.). The eluent from the column is then neutralized with a tertiary amine, such as tributylamine (TBA). The mixture is then lyophilized to obtain the tertiary amine salt of the N-sulfated hexosamine-containing polysaccharide. In addition, to generate the tertiary amine salt of the N-sulfated hexosamine-containing polysaccharide by batch ion-exchange, the N-sulfated hexosamine-containing polysaccharide in double distilled water is mixed with roughly the same volume of an ion exchange resin at refrigerated temperature (2° C. to 8° C.) with constant stirring. The mixture is then filtered and neutralized by the addition of the tertiary amine, such as tributylamine (TBA). The mixture is then lyophilized to obtain the tertiary amine salt of the N-sulfated hexosamine-containing polysaccharide.

Alternatively, the sodium salt of the N-sulfated hexosamine-containing polysaccharide can be converted to a quaternary amine salt, preferably a long chain quatenary amine salt. In this embodiment, a quaternary amine such as cetylpyridium chloride (CPC), benzethonium or cetyltrimethylammonium bromide, is dissolved in double distilled water to make about a 0.1% to about a 15% solution and, more preferably, about a 1% to about 10% solution. The quatenary amine salt solution is then added until no further precipitation is formed. The precipitate is collected by centrifugation or filtration, and lyophilized to obtain the quaternary amine salt of the N-sulfated hexosamine-containing polysaccharide.

In step two of the above process, a first solution is prepared by contacting the amine salt of the N-sulfated hexosamine-containing polysaccharide with any type of acid. In a preferred embodiment, the acid is a mineral acid such as sulfuric acid, hydrochloric acid, or phosphoric acid, or any combination of these acids. In a preferred embodiment, the acid is present in about 0.1 to about 100 molar equivalence of N-sulfate mols in the N-sulfated hexosamine-containing polysaccharide and, more preferably, in about 2 to about 50 molar equivalence of N-sulfate mols in the N-sulfated hexosamine-containing polysaccharide. Thereafter, the first solution is contacted with an N,N′-carbodiimide to form a second solution. Examples of suitable N,N′-carbodiimides include, but are not limited to, the following: diisopropylcarbodiimide (DIC); dicyclohexylcarbodiimide (DCC); 3-ethyl-[(3-dimethylaminopropyl) carbodiimide hydrochloride (EDC) and 1-cyclohexyl-3-(2-morpholoethyl)carbodiimide p-toluene sulfonate (CMC). Typically, the N,N′-carbodiimide is present in an amount that is 0.1 to 0.99 normal equivalence to the amount of acid used. In a preferred embodiment, DIC is the N,N′-carbodiimide used.

The second step of the above process is preferably carried out in an aprotic solvent. Suitable aprotic solvents include, but are not limited to, the following: dimethylformamide (DMF), dimethylsulfoxide (DMSO), dichloromethane (DCM) and pyridine (Py). In a presently preferred embodiment, dimethylformamide is employed as the solvent. In addition, the second step is preferably carried out at a temperature ranging from about minus 80° C. to about 100° C. and, more preferably, at a temperature of about 0° C. to 60° C.

After the addition of the N,N′-carbodiimide, the second solution is allowed to stand for a period of about 0.1 to 50 hours and, more preferably, for a period of about 1 to 24 hours. Thereafter, one to five volumes of dichloromethane and, more preferably two to three volumes of dichloromethane, and one half to two volumes of NaOH solution in about 0.1 to 3.0 N NaOH and, more preferably, about 0.5 to 2.0 N NaOH are added to the second solution. The pH of the second solution is adjusted to an alkaline pH and, more preferably, to a pH above 12. The aqueous phase is separated from organic phase by centrifugation or other means of phase separation. The aqueous solution is allowed to stand for a period of about 0.1 to 1.0 hour at a temperature of about 0° C. to 25° C. Thereafter, the pH of the aqueous solution is lowered to a pH of about 4.0 to about 7.0 and, more preferably, to a pH of about 6.0.

In the third step of the above process, applicable primarily to heparinoids, those hexosamine residues of the hexosamine-containing polysaccharide that have lost their sulfate groups during the transfer reaction are further N-sulfated by contacting the product of the second reaction with a sulfating agent. In a presently preferred embodiment, the N-sulfation is carried out according to the method of Lloyd, A. G., et al., Biochem. Pharmacol., 20:637-648.

Those of skill in the art will readily appreciate that the resulting N-sulfated hexosamine-containing polysaccharides can be subjected to further purification procedures. Such procedures include, but are not limited to, gel permeation chromatography, ultrafiltration, hydrophobic interaction chromatography, affinity chromatography, ion exchange chromatography, etc. Moreover, the molecular weight characteristics of the N-sulfated hexosamine-containing polysaccharide compounds of the present invention can be determined using standard techniques known to and used by those of skill in the art as described above. In a preferred embodiment, the molecular weight characteristics of the N-sulfated hexosamine-containing polysaccharide compounds of the present invention are determined by high performance size exclusion chromatography. Moreover, the structure of product of the N→O sulfate reaction can be analyzed by H-1 and C13 NMR, and disaccharide composition analysis, etc.

The compositions produced by the method disclosed in this invention are candidates for pharmaceutical use, such as treating and preventing cardiovascular disease or cancer. The compositions demonstrate various properties that can be appreciated by those skilled in the art that they are effective candidates for such pharmaceutical use.

### EXAMPLES

The following examples are offered to illustrate, but not to limit the present invention.

The following examples illustrate experimental protocols which can be used to cause N-sulfate migration to adjacent or near-by hydroxyl groups in hexosamine-containing polysaccharides. As noted above, in a presently preferred embodiment of the process of the present invention, the chemical reactions leading to the preparation of the O-sulfated hexosamine-containing polysaccharides are: (1) conversion of sodium salts of the N-sulfated hexosamine-containing polysaccharide into amine salts, and (2) induction of N→O-sulfate migration from the N-position to the O-position of the hexosamine-containing polysaccharide by carbodiimides.

### Example 1

### Induction of N→O Sulfate Transfer in Pyridinium Heparin by Diisopropylcarbodiimide (DIC) and Sulfuric Acid

### Example 1-A

Step 1: Conversion of Heparin to a Pyridinium Salt

Heparin (1 gram) was dissolved in 20 milliliters of distilled water. A Dowex 50W-X8 ion exchange column (20-50 mesh, H⁺ form) was equilibrated with distilled water at a temperature range of about 2° C. to about 4° C. The heparin solution was washed through the Dowex column with distilled water and the eluent was collected in a beaker. The pH of the solution was adjusted to a pH of 6.0 to 6.5 by addition of pyridine. The solution was lyophilized to obtain 1.05 gram of pyridinium-heparin (Py-heparin).

Step 2: Induction of N→O Sulfate Transfer in Pyridinium Heparin by Diisopropylcarbodiimide (DIC) and Sulfuric Acid

One hundred and fifty milligrams of Py-heparin were dissolved in 6 milliliters of DMF. The solution was cooled to about 2° C. to about 4° C. in an ice-water bath. Fifty three microliters of concentrated sulfuric acid was added to the solution with stirring. DIC (123 microliters) was added to this mixture. The reaction was stirred for 4 hours at about 2° C. to about 4° C. in an ice-water bath. At the end of the reaction period, the reaction solution was added to a mixture of 15 ml of dichloromethane (DCM) and 5 ml of 1N aqueous sodium hydroxide. After thorough mixing, the aqueous phase was separated from the organic phase by centrifugation at 1000 rpm in a Thermo Centra CL-2 centrifuge. The aqueous phase was removed from the centrifuge tube and kept at ambient temperature for about 30 minutes. The pH of the aqueous solution was then adjusted to about pH 6. The modified heparin formed in the N→O sulfate transfer reaction was isolated by ultrafiltration on a Millipore Centriprep apparatus with a sequence of 6 changes of water following the manufacturer's protocol. After ultrafiltration, the final volume was 5 ml.

Step 3: Re—N-sulfation of the Modified Heparin from Step 2

The modified heparin was further N-sulfated by adding sodium carbonate (0.5 gram) and pyridinium sulfur trioxide (0.3 gram) to the solution formed in Step 2. The reaction was carried out at 55° C. for about 6 hours. After the N-sulfation was complete, the re-N-sulfated product was isolated by untrafiltration using same Centroprep apparatus described in Step2., and lyophilized to yield final product.

### Example 1-B

Step 1: Conversion of Heparin to a Pyridinium Salt. Same as Step 3 in Example 1-A

Step 2: Induction of N→O Sulfate Transfer in Pyridinium Heparin by Diisopropylcarbodiimide (DIC) and Sulfuric Acid

One hundred and fifty milligrams of Py-heparin were dissolved in 6 milliliters of DMF. The solution was cooled to about 2° C. to about 4° C. in an ice-water bath. Fifty three microliters of concentrated sulfuric acid was added to the solution with stirring. DIC (123 microliters) was added to this mixture. The reaction was stirred for 24 hours at about 2° C. to about 4° C. in an ice-water bath. At the end of the reaction period, the reaction solution was added to a mixture of 15 ml of dichloromethane (DCM) and 5 ml of 1N aqueous sodium hydroxide. After thorough mixing, the aqueous phase was separated from the organic phase by centrifugation at 1000 rpm in a Thermo Centra CL-2 centrifuge. The aqueous phase was removed from the centrifuge tube and kept at ambient temperature for about 30 minutes. The pH of the aqueous solution was then adjusted to about pH 6. The modified heparin formed in the N→O sulfate transfer reaction was isolated by ultrafiltration on a Millipore Centriprep apparatus with a sequence of 6 changes of water following the manufacturer's protocol. After ultrafiltration, the final volume was 5 ml. H1 NMR analysis of the product showed about 90% of N-sulfate was lost from glucosamine residues of the heparin.

Step 3: Re—N-sulfation of the Modified Heparin from Step 2. Same as Step 3 in Example 1-A

Analytical Result:

H1 NMR analysis of the product showed that at least about 90% of glucosamine residues of the heparin contain both 3-O-sulfate and N-sulfate.

### Example 1-C

Step 1: Conversion of Heparin to a Pyridinium Salt. Same as Step 1 in Example 1-A

Step 2: Induction of N→O Sulfate Transfer in Pyridinium Heparin by Diisopropylcarbodiimide (DIC) and Sulfuric Acid

One hundred and fifty milligrams of Py-heparin were dissolved in 6 milliliters of DMF. The solution was cooled to about 2° C. to about 4° C. in an ice-water bath. Fifty three microliters of concentrated sulfuric acid was added to the solution with stirring. DIC (123 microliters) was added to this mixture. The reaction was stirred for 4 hours at ambient temperature. At the end of the reaction period, the reaction solution was added to a mixture of 15 ml of dichloromethane (DCM) and 5 ml of 1N aqueous sodium hydroxide. After thorough mixing, the aqueous phase was separated from the organic phase by centrifugation at 1000 rpm in a Thermo Centra CL-2 centrifuge. The aqueous phase was removed from the centrifuge tube and kept at ambient temperature for about 30 minutes. The pH of the aqueous solution was then adjusted to about pH 6. The modified heparin formed in the N→O sulfate transfer reaction was isolated by ultrafiltration on a Millipore Centriprep apparatus with a sequence of 6 changes of water following the manufacturer's protocol. After ultrafiltration, the final volume was 5 ml. H1 NMR analysis of the product showed about 60% to 70% of N-sulfate was lost from glucosamine residues of the heparin.

Step 3: Re—N-sulfation of the Modified Heparin from Step 2. Same as Step 3 in Example 1-A

### Example 2

### Induction of N→O Sulfate Transfer in Pyridinium Heparin by Diisopropylcarbodiimide (DIC) and Hydrochloric acid

### Example 2-A

Step 1: Same as Step 1 in Example 1-A

Step 2: Induction of N→O Sulfate Transfer in Pyridinium Heparin by Diisopropylcarbodiimide (DIC) and Hydrochloric Acid

One hundred and fifty milligrams of Py-heparin were dissolved in 6 milliliters of DMF. The solution was cooled to about 2° C. to about 4° C. in an ice water bath. Five hundred microliters of 4M HCl in dioxane was added to the solution with stirring. DIC (185 microliters) was added to this mixture. The reaction was carried out at about 2° C. to about 4° C. in an ice-water bath for 4 hours with stirring. At the end of the reaction period, the reaction mixture was added to a mixture of 15 ml of dichloromethane (DCM) and 5 ml of 1N sodium hydroxide. After thoroughly mixing, the aqueous phase was separated from organic phase by centrifugation at 1000 rpm in a Thermo Centra CL-2 centrifuge. The aqueous phase was removed from the centrifuge tube and kept at ambient temperature for about 30 minutes. The pH of the aqueous solution was then adjusted to about pH 6. The structurally modified heparin formed in the N→O sulfate transfer reaction was isolated by ultrafiltration using Millipore Centriprep apparatus a sequence of 6 changes of water following the manufacturer's protocol. After ultrafiltration, the final volume is 5 ml. H1-nmr analysis of one the example showed about 90% of N-sulfate was lost from N-sulfated glucosamine residues of heparin.

Step 3: Same as Step 3 in Example 1-A

### Example 2-B

Step 1: Same as Step 1 in Example 1-A

Step 2: Induction of N→O Sulfate Transfer in Pyridinium Heparin by Diisopropylcarbodiimide (DIC) and Hydrochloric Acid

One hundred and fifty milligrams of Py-heparin were dissolved in 6 milliliters of DMF. The solution was cooled to about 2° C. to about 4° C. in an ice water bath. Five hundred microliters of 4M HCl in dioxane was added to the solution with stirring. DIC (123 microliters) was added to this mixture. The reaction was carried out at about 55° C. to about 60° C. in a water bath for 4 hours with stirring. At the end of the reaction period, the reaction mixture was cooled to ambient temperature and added to a mixture of 15 ml of dichloromethane (DCM) and 5 ml of 1N sodium hydroxide. After thoroughly mixing, the aqueous phase was separated from organic phase by centrifugation at 1000 rpm in a Thermo Centra CL-2 centrifuge. The aqueous phase was removed from the centrifuge tube and kept at ambient temperature for about 30 minutes. The pH of the aqueous solution was then adjusted to about pH 6. The structurally modified heparin formed in the N→O sulfate transfer reaction was isolated by ultrafiltration using a Millipore Centriprep apparatus with a sequence of 6 changes of water following the manufacturer's protocol. After ultrafiltration, the final volume is 5 ml.

Step 3: Same as Step 3 in Example 1-A

### Example 3

### Induction of N→O Sulfate Transfer in Pyridinium Heparin by Diisopropylcarbodiimide (DIC), and Both Sulfuric Acid and Hydrochloric Acid

Step 1: Same as Step 1 in Example 1-A

Step 2: Induction of N→O Sulfate Transfer in Py-Heparin by Diisopropylcarbodiimide (DIC), and Both Sulfuric Acid and Hydrochloric Acid

One hundred and fifty milligrams of Py-heparin were dissolved in 6 milliliters of DMF. The solution was cooled to about 2° C. to about 4° C. in an ice water bath. Fifty three microliters of concentrated sulfuric acid was added to the solution with stirring. DIC (123 microliters) was added to this mixture. The reaction was stirred for 2 hours at about 2° C. to about 4° C. in an ice-water bath. Then, two hundred microliters of 4M HCl in dioxane was added to the solution with stirring. DIC (123 microliters) was added to this mixture. The reaction was carried out at about 2° C. to about 4° C. in an ice-water bath for additional 4 hours with stirring. At the end of the reaction period, the reaction mixture was added to a mixture of 15 ml of dichloromethane (DCM) and 5 ml of 1N sodium hydroxide. After thoroughly mixing, the aqueous phase was separated from organic phase by centrifugation at 1000 rpm in a Thermo Centra CL-2 centrifuge. The aqueous phase was removed from the centrifuge tube and kept at ambient temperature for about 30 minutes. The pH of the aqueous solution was then adjusted to about pH 6. The structurally modified heparin formed in the N→O sulfate transfer reaction was isolated by ultrafiltration using Millipore Centriprep apparatus a sequence of 6 changes of water following the manufacturer's protocol. After ultrafiltration, the final volume is 5 ml.

Step 3: Same as Step 3 in Example 1-A

### Example 4

### Induction of N→O Sulfate Transfer in Tributylamine (TBA) Heparin by Diisopropylcarbodiimide (DIC) and Sulfuric Acid

Step 1: Conversion of Heparin to a Tributylamine Salt

Heparin (1 gram) was dissolved in 20 milliliters of distilled water. A Dowex 50W-X8 ion exchange column (20-50 mesh, H⁺ form) was equilibrated with distilled water at a temperature range of about 2° C. to about 4° C. The heparin solution was washed through the Dowex column with distilled water and the eluent was collected in a beaker and mixed with tributylamine (0.3 grams). The pH of the solution was adjusted to a pH of 6.0 to 6.5 by addition of tributylamine. The solution was lyophilized to obtain 1.6 gram of tributylamine-heparin (TBA-heparin).

Step 2: Induction of N→O Sulfate Transfer in TBA Heparin by Diisopropylcarbodiimide (DIC) and Sulfuric Acid

Two hundred and eighteen milligrams of TBA-heparin were dissolved in 6 milliliters of DMF. The solution was cooled to about 2° C. to about 4° C. in an ice-water bath. Fifty three microliters of concentrated sulfuric acid was added to the solution with stirring. DIC (123 microliters) was added to this mixture. The reaction was stirred for 6 hours at about 2° C. to about 4° C. in an ice-water bath. At the end of the reaction period, the reaction solution was added to a mixture of 15 ml of dichloromethane (DCM) and 5 ml of 1N aqueous sodium hydroxide. After thorough mixing, the aqueous phase was separated from the organic phase by centrifugation at 1000 rpm in a Thermo Centra CL-2 centrifuge. The aqueous phase was removed from the centrifuge tube and kept at ambient temperature for about 30 minutes. The pH of the aqueous solution was then adjusted to about pH 6. The modified heparin formed in the N→O sulfate transfer reaction was isolated by ultrafiltration on a Millipore Centriprep apparatus with a sequence of 6 changes of water following the manufacturer's protocol. After ultrafiltration, the final volume was 5 ml.

Step 3: Same as Step 3 in Example 1-A

### Example 5

### Induction of N→O Sulfate Transfer in Cetylpyridinium Heparin (CPy-Heparin) by Diisopropylcarbodiimide (DIC) and Sulfuric Acid in Dichloromethane (DCM)

Step 1: Conversion of Heparin to a Cetylpyridinium Salt

Heparin (1.25 grams) was dissolved in 10 milliliters of 10 mM sodium sulfate aqueous solution. Cetylpyridium chloride (CPC) was dissolved in water to form a 10% solution. Three milliliters of the CPC solution was added to the heparin solution. The mixture was centrifuged to form a pellet. The pellet was washed twice with distilled water. The pellet was lyophilized to generate 2.2 grams of CPy-heparin.

Step 2: Induction of N→O Sulfate Transfer in CPy-Heparin by Diisopropylcarbodiimide (DIC) and Sulfuric Acid in DCM

Two hundred and eighty milligrams of CPy-heparin were dissolved in 6 milliliters of DMF. The solution was cooled to about 2° C. to about 4° C. in an ice water bath. Fifty three microliters of concentrated sulfuric acid was added to the solution with stirring. DIC (123 microliters) was added to this mixture. The reaction was stirred for 4 hours at about 2° C. to about 4° C. in an ice-water bath. At the end of the reaction period, the reaction mixture was added to a mixture of 10 ml of DCM and 5 ml of 1N sodium hydroxide. After thoroughly mixing, the aqueous phase was separated from organic phase by centrifugation at 1000 rpm in a Thermo Centra CL-2 centrifuge. The aqueous phase was removed from the centrifuge tube and kept at ambient temperature for about 30 minutes. The pH of the aqueous solution was then adjusted to about pH 6. The structurally modified heparin formed in the N→O sulfate transfer reaction was isolated by ultrafiltration using Millipore Centriprep apparatus with a sequence of 6 changes of water following the manufacturer's protocol. After ultrafiltration, the final volume is 5 ml.

Step 3: Same as Step 3 in Example 1-A

### Example 6

### Induction of N→O Sulfate Transfer in Pyridinium De-O-Sulfated Heparin (De-OSu-Heparin) by Diisopropylcarbodiimide (DIC) and Sulfuric Acid

Step 1: Conversion of De-OS-Heparin to a Pyridinium Salt

De-OS-Heparin (1 gram) was dissolved in 20 milliliters of distilled water. A Dowex 50W-X8 ion exchange column (20-50 mesh, H⁺ form) was equilibrated with distilled water at a temperature range of about 2° C. to about 4° C. The de-O-sulfated heparin solution was washed through the Dowex column with distilled water and the eluent was collected in a beaker. The pH of the solution was adjusted to a pH of 6.0 to 6.5 by addition of pyridine. The solution was lyophilized to obtain 0.95 gram of pyridinimu de-o-sulfated heparin (Py-DeOS-heparin).

Step 2: Induction of N→O Sulfate Transfer in Pyridinium De-O-Sulfated Heparin (Py-DeOS-heparin) by Diisopropylcarbodiimide (DIC) and Sulfuric Acid

One hundred and fifteen milligrams of Py-DeOS-heparin were dissolved in 6 milliliters of DMF. The solution was cooled to about 2° C. to about 4° C. in an ice-water bath. One hundred and sixty microliters of concentrated sulfuric acid was added to the solution with stirring. DIC (370 microliters) was added to this mixture. The reaction was stirred for 24 hours at about 2° C. to about 4° C. in an ice-water bath. At the end of the reaction period, the reaction solution was added to a mixture of 15 ml of dichloromethane (DCM) and 5 ml of 1N aqueous sodium hydroxide. After thorough mixing, the aqueous phase was separated from the organic phase by centrifugation at 1000 rpm in a Thermo Centra CL-2 centrifuge. The aqueous phase was removed from the centrifuge tube and kept at ambient temperature for about 30 minutes. The pH of the aqueous solution was then adjusted to about pH 6. The modified de-O-sulfated heparin formed in the N→O sulfate transfer reaction was isolated by ultrafiltration on a Millipore Centriprep apparatus with a sequence of 6 changes of water following the manufacturer's protocol. After ultrafiltration, the final volume was 5 ml.

Step 3: Same as Step 3 in Example 1.-A

### Example 7

### Induction of N→O Sulfate Transfer in Pyridinium Heparan Sulfate (Py-HS) by Diisopropylcarbodiimide (DIC) and Sulfuric Acid

Step 1: Conversion of Heparan Sulfate to a Pyridinium Salt

Heparan sulfate (0.5 gram) was dissolved in 10 milliliters of distilled water. A Dowex 50W-X8 ion exchange column (20-50 mesh, H⁺ form) was equilibrated with distilled water at a temperature range of about 2° C. to about 4° C. The heparan sulfate solution was washed through the Dowex column with distilled water and the eluent was collected in a beaker. The pH of the solution was adjusted to a pH of 6.0 to 6.5 by addition of pyridine. The solution was lyophilized to obtain 0.45 gram of pyridinium heparan sulfate (Py-HS).

Step 2: Induction of N→O Sulfate Transfer in Pyridinium Heparan Sulfate (Py-HS) by Diisopropylcarbodiimide (DIC) and Sulfuric Acid

One hundred and fifteen milligrams of Py-HS were dissolved in 6 milliliters of DMF. The solution was cooled to about 2° C. to about 4° C. in an ice-water bath. One hundred and six microliters of concentrated sulfuric acid was added to the solution with stirring. DIC (230 microliters) was added to this mixture. The reaction was stirred for 8 hours at about 2° C. to about 4° C. in an ice-water bath. At the end of the reaction period, the reaction solution was added to a mixture of 15 ml of dichloromethane (DCM) and 5 ml of 1N aqueous sodium hydroxide. After thorough mixing, the aqueous phase was separated from the organic phase by centrifugation at 1000 rpm in a Thermo Centra CL-2 centrifuge. The aqueous phase was removed from the centrifuge tube and kept at ambient temperature for about 30 minutes. The pH of the aqueous solution was then adjusted to about pH 6. The modified heparan sulfate formed in the N→O sulfate transfer reaction was isolated by ultrafiltration on a Millipore Centriprep apparatus with a sequence of 6 changes of water following the manufacturer's protocol. After ultrafiltration, the final volume was 5 ml.

Step 3: Same as Step 3 in Example 1-A

### Example 8

### Induction of N→O Sulfate Transfer in Pyridinium De-acetylated, N-sulfated Heparan Sulfate (DAc-NS-HS) by Diisopropylcarbodiimide (DIC) and Sulfuric Acid

Step 1: Conversion of DAc-NS-HS to a Pyridinium Salt

DAc-NS-HS (0.5 gram) was dissolved in 10 milliliters of distilled water. A Dowex 50W-X8 ion exchange column (20-50 mesh, H⁺ form) was equilibrated with distilled water at a temperature range of about 2° C. to about 4° C. The DAc-NS-HS solution was washed through the Dowex column with distilled water and the eluent was collected in a beaker. The pH of the solution was adjusted to a pH of 6.0 to 6.5 by addition of pyridine. The solution was lyophilized to obtain 0.45 gram of pyridinium-DAc-NS-HS (Py-DAc-NS-HS).

Step 2: Induction of N→O Sulfate Transfer in Py-DAc-NS-HS by Diisopropylcarbodiimide (DIC) and Sulfuric Acid

One hundred and fifteen milligrams of Py-DAc-NS-HS were dissolved in 6 milliliters of DMF. The solution was cooled to about 2° C. to about 4° C. in an ice-water bath. One hundred and sixty microliters of concentrated sulfuric acid was added to the solution with stirring. DIC (370 microliters) was added to this mixture. The reaction was stirred for 6 hours at about 2° C. to about 4° C. in an ice-water bath. At the end of the reaction period, the reaction solution was added to a mixture of 15 ml of dichloromethane (DCM) and 5 ml of 1N aqueous sodium hydroxide. After thorough mixing, the aqueous phase was separated from the organic phase by centrifugation at 1000 rpm in a Thermo Centra CL-2 centrifuge. The aqueous phase was removed from the centrifuge tube and kept at ambient temperature for about 30 minutes. The pH of the aqueous solution was then adjusted to about pH 6. The modified DAc-NS-HS formed in the N→O sulfate transfer reaction was isolated by ultrafiltration on a Millipore Centriprep apparatus with a sequence of 6 changes of water following the manufacturer's protocol. After ultrafiltration, the final volume was 5 ml.

Step 3: Same as Step 3 in Example 1-A

### Example 9

### Induction of O→O Sulfate Transfer in Pyridinium Dermatan Sulfate by Diisopropylcarbodiimide (DIC) and Sulfuric Acid

Step 1: Conversion of Dermatan Sulfate to a Pyridinium Salt

Dermatan sulfate (0.5 gram) was dissolved in 10 milliliters of distilled water. A Dowex 50W-X8 ion exchange column (20-50 mesh, H⁺ form) was equilibrated with distilled water at a temperature range of about 2° C. to about 4° C. The dermatan sulfate solution was washed through the Dowex column with distilled water and the eluent was collected in a beaker. The pH of the solution was adjusted to a pH of 6.0 to 6.5 by addition of pyridine. The solution was lyophilized to obtain 0.45 gram of pyridinium dermatan sulfate (Py-DS).

Step 2: Induction of O→O Sulfate Transfer in Pyridinium Dermatan Sulfate (Py-DS) by Diisopropylcarbodiimide (DIC) and Sulfuric Acid

One hundred and fifteen milligrams of Py-DS were dissolved in 6 milliliters of DMF. The solution was cooled to about 2° C. to about 4° C. in an ice-water bath. One hundred and sixty microliters of concentrated sulfuric acid was added to the solution with stirring. DIC (370 microliters) was added to this mixture. The reaction was stirred for 8 hours at ambient temperature. At the end of the reaction period, the reaction solution was added to a mixture of 15 ml of dichloromethane (DCM) and 5 ml of 1N aqueous sodium hydroxide. After thorough mixing, the aqueous phase was separated from the organic phase by centrifugation at 1000 rpm in a Thermo Centra CL-2 centrifuge. The aqueous phase was removed from the centrifuge tube and kept at ambient temperature for about 30 minutes. The pH of the aqueous solution was then adjusted to about pH 6. The modified dermatan sulfate formed in the O→O sulfate transfer reaction was isolated by ultrafiltration on a Millipore Centriprep apparatus with a sequence of 6 changes of water following the manufacturer's protocol. After ultrafiltration, the final product was obtained by lyophilization.

### Example 10

### Induction of N→O Sulfate Transfer in Pyridinium De-acetylated, N-sulfated Dermatan Sulfate (DAc-NS-DS) by Diisopropylcarbodiimide (DIC) and Sulfuric Acid

Step 1: Conversion of DAc-NS-DS to a Pyridinium Salt

DAc-NS-DS (0.5 gram) was dissolved in 10 milliliters of distilled water. A Dowex 50W-X8 ion exchange column (20-50 mesh, H⁺ form) was equilibrated with distilled water at a temperature range of about 2° C. to about 4° C. The DAc-NS-DS solution was washed through the Dowex column with distilled water and the eluent was collected in a beaker. The pH of the solution was adjusted to a pH of 6.0 to 6.5 by addition of pyridine. The solution was lyophilized to obtain 0.45 gram of pyridinium-DAc-NS-DS (Py-DAc-NS-DS).

Step 2: Induction of N→O Sulfate Transfer in Py-DAc-NS-DS by Diisopropylcarbodiimide (DIC) and Sulfuric Acid

One hundred and fifteen milligrams of Py-DAc-NS-DS were dissolved in 6 milliliters of DMF. The solution was cooled to about 2° C. to about 4° C. in an ice-water bath. One hundred and sixty microliters of concentrated sulfuric acid was added to the solution with stirring. DIC (370 microliters) was added to this mixture. The reaction was stirred for 6 hours at about 2° C. to about 4° C. in an ice-water bath. At the end of the reaction period, the reaction solution was added to a mixture of 15 ml of dichloromethane (DCM) and 5 ml of 1N aqueous sodium hydroxide. After thorough mixing, the aqueous phase was separated from the organic phase by centrifugation at 1000 rpm in a Thermo Centra CL-2 centrifuge. The aqueous phase was removed from the centrifuge tube and kept at ambient temperature for about 30 minutes. The pH of the aqueous solution was then adjusted to about pH 6. The modified DAc-NS-DS formed in the N→O sulfate transfer reaction was isolated by ultrafiltration on a Millipore Centriprep apparatus with a sequence of 6 changes of water following the manufacturer's protocol. After ultrafiltration, the final volume was 5 ml.

Step 3: Same as Step 3 in Example 1-A

### Example 11

### Induction of N→O Sulfate Transfer in Pyridinium N-Sulfated Chitosan (NS-Chitosan) by Diisopropylcarbodiimide (DIC) and Sulfuric Acid

Step 1: Conversion of NS-Chitosan to a Pyridinium Salt

NS-Chitosan (0.5 gram) was dissolved in 10 milliliters of distilled water. A Dowex 50W-X8 ion exchange column (20-50 mesh, H⁺ form) was equilibrated with distilled water at a temperature range of about 2° C. to about 4° C. The NS-chitosan solution was washed through the Dowex column with distilled water and the eluent was collected in a beaker. The pH of the solution was adjusted to a pH of 6.0 to 6.5 by addition of pyridine. The solution was lyophilized to obtain 0.46 gram of pyridinium-NS-chitosan (Py-NS-chitosan).

Step 2: Induction of N→O Sulfate Transfer in Py-NS-chitosan by Diisopropylcarbodiimide (DIC) and Sulfuric Acid

One hundred and thirty five milligrams of Py-NS-chitosan were dissolved in 6 milliliters of DMF. The solution was cooled to about 2° C. to about 4° C. in an ice-water bath. Two hundred and ten microliters of concentrated sulfuric acid was added to the solution with stirring. DIC (246 microliters) was added to this mixture. The reaction was stirred for 24 hours at about 2° C. to about 4° C. in an ice-water bath. At the end of the reaction period, the reaction solution was added to a mixture of 15 ml of dichloromethane (DCM) and 5 ml of 1N aqueous sodium hydroxide. After thorough mixing, the aqueous phase was separated from the organic phase by centrifugation at 1000 rpm in a Thermo Centra CL-2 centrifuge. The aqueous phase was removed from the centrifuge tube and kept at ambient temperature for about 30 minutes. The pH of the aqueous solution was then adjusted to about pH 6. The modified NS-chitosan formed in the N→O sulfate transfer reaction was isolated by ultrafiltration on a Millipore Centriprep apparatus with a sequence of 6 changes of water following the manufacturer's protocol. After ultrafiltration, the final volume was 5 ml.

Step 3: Same as Step 3 in Example 1-A

### Example 12

### Induction of N→O Sulfate Transfer in Pyridinium N-Sulfated Chitosan (NS-Chitosan) by Diisopropylcarbodiimide (DIC) and Hydrochloric Acid

Step 1: Conversion of NS-Chitosan to a Pyridinium Salt. Same as Step 1 in Example 11.

Step 2: Induction of N→O Sulfate Transfer in Py-NS-chitosan by Diisopropylcarbodiimide (DIC) and Hydrochloric Acid

One hundred and thirty five milligrams of Py-NS-chitosan were dissolved in 6 milliliters of DMF. The solution was cooled to about 2° C. to about 4° C. in an ice-water bath. Five hundred microliters of 4M HCl in dioxane was added to the solution with stirring. DIC (246 microliters) was added to this mixture. The reaction was stirred for 16 hours at about 2° C. to about 4° C. in an ice-water bath. At the end of the reaction period, the reaction solution was added to a mixture of 15 ml of dichloromethane (DCM) and 5 ml of 1N aqueous sodium hydroxide. After thorough mixing, the aqueous phase was separated from the organic phase by centrifugation at 1000 rpm in a Thermo Centra CL-2 centrifuge. The aqueous phase was removed from the centrifuge tube and kept at ambient temperature for about 30 minutes. The pH of the aqueous solution was then adjusted to about pH 6. The modified NS-chitosan formed in the N→O sulfate transfer reaction was isolated by ultrafiltration on a Millipore Centriprep apparatus with a sequence of 6 changes of water following the manufacturer's protocol. After ultrafiltration, the final volume was 5 ml.

Step 3: Same as Step 3 in Example 1-A

### Example 13

### Induction of O→O Sulfate Transfer in Pyridinium Chondroitin Sulfate by Diisopropylcarbodiimide (DIC) and Sulfuric Acid

Step 1: Conversion of Chondroitin Sulfate to a Pyridinium Salt

Chondroitin sulfate (0.5 gram) was dissolved in 10 milliliters of distilled water. A Dowex 50W-X8 ion exchange column (20-50 mesh, H⁺ form) was equilibrated with distilled water at a temperature range of about 2° C. to about 4° C. The chondroitin sulfate solution was washed through the Dowex column with distilled water and the eluent was collected in a beaker. The pH of the solution was adjusted to a pH of 6.0 to 6.5 by addition of pyridine. The solution was lyophilized to obtain 0.45 gram of pyridinium chondroitin sulfate (Py-DS).

Step 2: Induction of O→O Sulfate Transfer in Pyridinium Chondroitin Sulfate (Py-CS) by Diisopropylcarbodiimide (DIC) and Sulfuric Acid

One hundred and fifteen milligrams of Py-CS were dissolved in 6 milliliters of DMF. The solution was cooled to about 2° C. to about 4° C. in an ice-water bath. One hundred and sixty microliters of concentrated sulfuric acid was added to the solution with stirring. DIC (370 microliters) was added to this mixture. The reaction was stirred for 8 hours at ambient temperature. At the end of the reaction period, the reaction solution was added to a mixture of 15 ml of dichloromethane (DCM) and 5 ml of 1N aqueous sodium hydroxide. After thorough mixing, the aqueous phase was separated from the organic phase by centrifugation at 1000 rpm in a Thermo Centra CL-2 centrifuge. The aqueous phase was removed from the centrifuge tube and kept at ambient temperature for about 30 minutes. The pH of the aqueous solution was then adjusted to about pH 6. The modified chondroitin sulfate formed in the O→O sulfate transfer reaction was isolated by ultrafiltration on a Millipore Centriprep apparatus with a sequence of 6 changes of water following the manufacturer's protocol. After ultrafiltration, the final product was obtained by lyophilization.

### Example 14

### Induction of N→O Sulfate Transfer in Pyridinium De-acetylated, N-sulfated Chondroitin Sulfate (DAc-NS-CS) by Diisopropylcarbodiimide (DIC) and Sulfuric Acid

Step 1: Conversion of DAc-NS-CS to a Pyridinium Salt

DAC-NS-CS (0.5 gram) was dissolved in 10 milliliters of distilled water. A Dowex 50W-X8 ion exchange column (20-50 mesh, H⁺ form) was equilibrated with distilled water at a temperature range of about 2° C. to about 4° C. The DAc-NS-CS solution was washed through the Dowex column with distilled water and the eluent was collected in a beaker. The pH of the solution was adjusted to a pH of 6.0 to 6.5 by addition of pyridine. The solution was lyophilized to obtain 0.45 gram of pyridinium-DAc-NS-CS (Py-DAc-NS-CS).

Step 2: Induction of N→O Sulfate Transfer in Py-DAc-NS-CS by Diisopropylcarbodiimide (DIC) and Sulfuric Acid

One hundred and fifteen milligrams of Py-DAc-NS-CS were dissolved in 6 milliliters of DMF. The solution was cooled to about 2° C. to about 4° C. in an ice-water bath. One hundred and sixty microliters of concentrated sulfuric acid was added to the solution with stirring. DIC (370 microliters) was added to this mixture. The reaction was stirred for 6 hours at about 2° C. to about 4° C. in an ice-water bath. At the end of the reaction period, the reaction solution was added to a mixture of 15 ml of dichloromethane (DCM) and 5 ml of 1N aqueous sodium hydroxide. After thorough mixing, the aqueous phase was separated from the organic phase by centrifugation at 1000 rpm in a Thermo Centra CL-2 centrifuge. The aqueous phase was removed from the centrifuge tube and kept at ambient temperature for about 30 minutes. The pH of the aqueous solution was then adjusted to about pH 6. The modified DAc-NS-CS formed in the N→O sulfate transfer reaction was isolated by ultrafiltration on a Millipore Centriprep apparatus with a sequence of 6 changes of water following the manufacturer's protocol. After ultrafiltration, the final volume was 5 ml.

Step 3: Same as Step 3 in Example 1-A

### Example 15

### Induction of N→O Sulfate Transfer in Pyridinium De-acetylated, N-sulfated Hyaluronic Acid (DAc-NS-Hya) by Diisopropylcarbodiimide (DIC) and Sulfuric Acid

Step 1: Conversion of DAc-NS-Hya to a Pyridinium Salt

DAc-NS-Hya (0.5 gram) was dissolved in 30 milliliters of distilled water. A Dowex 50W-X8 ion exchange column (20-50 mesh, H⁺ form) was equilibrated with distilled water at a temperature range of about 2° C. to about 4° C. The DAc-NS-Hya solution was washed through the Dowex column with distilled water and the eluent was collected in a beaker. The pH of the solution was adjusted to a pH of 6.0 to 6.5 by addition of pyridine. The solution was lyophilized to obtain 0.45 gram of pyridinium-DAc-NS-Hya (Py-DAc-NS-Hya).

Step 2: Induction of N→O Sulfate Transfer in Py-DAc-NS-Hya by Diisopropylcarbodiimide (DIC) and Sulfuric Acid

One hundred and fifteen milligrams of Py-DAc-NS-Hya were dissolved in 6 milliliters of DMF. The solution was cooled to about 2° C. to about 4° C. in an ice-water bath. One hundred and sixty microliters of concentrated sulfuric acid was added to the solution with stirring. DIC (370 microliters) was added to this mixture. The reaction was stirred for 6 hours at about 2° C. to about 4° C. in an ice-water bath. At the end of the reaction period, the reaction solution was added to a mixture of 15 ml of dichloromethane (DCM) and 5 ml of 1N aqueous sodium hydroxide. After thorough mixing, the aqueous phase was separated from the organic phase by centrifugation at 1000 rpm in a Thermo Centra CL-2 centrifuge. The aqueous phase was removed from the centrifuge tube and kept at ambient temperature for about 30 minutes. The pH of the aqueous solution was then adjusted to about pH 6. The modified DAc-NS-Hya formed in the N→O sulfate transfer reaction was isolated by ultrafiltration on a Millipore Centriprep apparatus with a sequence of 6 changes of water following the manufacturer's protocol. After ultrafiltration, the final volume was 5 ml.

Step 3: Same as Step 3 in Example 1-A

### Example 16

### Induction of N→O Sulfate Transfer in Pyridinium Epimerized De-Acetylated, N-sulfated K5 (EK5NS) by Diisopropylcarbodiimide (DIC) and Sulfuric Acid

Step 1: Conversion of EK5NS to a Pyridinium Salt (Same for Examples 16-A, 16-B, 16-C, and 16-D)

EK5NS (0.5 gram) was dissolved in 30 milliliters of distilled water. A Dowex 50W-X8 ion exchange column (20-50 mesh, H⁺ form) was equilibrated with distilled water at a temperature range of about 2° C. to about 4° C. The EK5NS solution was washed through the Dowex column with distilled water and the eluent was collected in a beaker. The pH of the solution was adjusted to a pH of 6.0 to 6.5 by addition of pyridine. The solution was lyophilized to obtain 0.45 gram of pyridinium-EK5NS (Py-EK5NS).

### Example 16-A

Step 2: Induction of N→O Sulfate Transfer in Py-EK5NS by Diisopropylcarbodiimide (DIC) and Sulfuric Acid

One hundred and fifteen milligrams of Py-EK5NS were dissolved in 6 milliliters of DMF. The solution was cooled to about 2° C. to about 4° C. in an ice-water bath. One hundred and six microliters of concentrated sulfuric acid was added to the solution with stirring. DIC (245 microliters) was added to this mixture. The reaction was stirred for 4 hours at about 2° C. to about 4° C. in an ice-water bath. At the end of the reaction period, the reaction solution was added to a mixture of 15 ml of dichloromethane (DCM) and 5 ml of 1N aqueous sodium hydroxide. After thorough mixing, the aqueous phase was separated from the organic phase by centrifugation at 1000 rpm in a Thermo Centra CL-2 centrifuge. The aqueous phase was removed from the centrifuge tube and kept at ambient temperature for about 30 minutes. The pH of the aqueous solution was then adjusted to about pH 6. The modified EK5NS formed in the N→O sulfate transfer reaction was isolated by ultrafiltration on a Millipore Centriprep apparatus with a sequence of 6 changes of water following the manufacturer's protocol. After ultrafiltration, the final volume was 5 ml.

### Example 16-B

Step 2: Induction of N→O Sulfate Transfer in EK5NS by Diisopropylcarbodiimide (DIC) and Sulfuric Acid

One hundred and fifteen milligrams of Py-EK5NS were dissolved in 6 milliliters of DMF. The solution was cooled to about 2° C. to about 4° C. in an ice-water bath. One hundred and six microliters of concentrated sulfuric acid was added to the solution with stirring. DIC (245 microliters) was added to this mixture. The reaction was stirred for 8 hours at about 2° C. to about 4° C. in an ice-water bath. At the end of the reaction period, the reaction solution was added to a mixture of 15 ml of dichloromethane (DCM) and 5 ml of 1N aqueous sodium hydroxide. After thorough mixing, the aqueous phase was separated from the organic phase by centrifugation at 1000 rpm in a Thermo Centra CL-2 centrifuge. The aqueous phase was removed from the centrifuge tube and kept at ambient temperature for about 30 minutes. The pH of the aqueous solution was then adjusted to about pH 6. The modified EK5NS formed in the N→O sulfate transfer reaction was isolated by ultrafiltration on a Millipore Centriprep apparatus with a sequence of 6 changes of water following the manufacturer's protocol. After ultrafiltration, the final volume was 5 ml.

### Example 16-C

Step 2: Induction of N→O Sulfate Transfer in EK5NS by Diisopropylcarbodiimide (DIC) and Sulfuric Acid

One hundred and fifteen milligrams of Py-EK5NS were dissolved in 6 milliliters of DMF. The solution was cooled to about 2° C. to about 4° C. in an ice-water bath. One hundred and six microliters of concentrated sulfuric acid was added to the solution with stirring. DIC (245 microliters) was added to this mixture. The reaction was stirred for 16 hours at about 2° C. to about 4° C. in an ice-water bath. At the end of the reaction period, the reaction solution was added to a mixture of 15 ml of dichloromethane (DCM) and 5 ml of 1N aqueous sodium hydroxide. After thorough mixing, the aqueous phase was separated from the organic phase by centrifugation at 1000 rpm in a Thermo Centra CL-2 centrifuge. The aqueous phase was removed from the centrifuge tube and kept at ambient temperature for about 30 minutes. The pH of the aqueous solution was then adjusted to about pH 6. The modified EK5NS formed in the N→O sulfate transfer reaction was isolated by ultrafiltration on a Millipore Centriprep apparatus with a sequence of 6 changes of water following the manufacturer's protocol. After ultrafiltration, the final volume was 5 ml.

### Example 16-D

Step 2: Induction of N→O Sulfate Transfer in EK5NS by Diisopropylcarbodiimide (DIC) and Sulfuric Acid

One hundred and fifteen milligrams of Py-EK5NS were dissolved in 6 milliliters of DMF. The solution was cooled to about 2° C. to about 4° C. in an ice-water bath. One hundred and six microliters of concentrated sulfuric acid was added to the solution with stirring. DIC (245 microliters) was added to this mixture. The reaction was stirred for 24 hours at about 2° C. to about 4° C. in an ice-water bath. At the end of the reaction period, the reaction solution was added to a mixture of 15 ml of dichloromethane (DCM) and 5 ml of 1N aqueous sodium hydroxide. After thorough mixing, the aqueous phase was separated from the organic phase by centrifugation at 1000 rpm in a Thermo Centra CL-2 centrifuge. The aqueous phase was removed from the centrifuge tube and kept at ambient temperature for about 30 minutes. The pH of the aqueous solution was then adjusted to about pH 6. The modified EK5NS formed in the N→O sulfate transfer reaction was isolated by ultrafiltration on a Millipore Centriprep apparatus with a sequence of 6 changes of water following the manufacturer's protocol. After ultrafiltration, the final volume was 5 ml.

Step 3: Examples 16-A, 16-B, 16-C and 16-D were re-N-sulfated as in Step 3 in Example 1-A

### Example 17

### Induction of N→O Sulfate Transfer in Pyridinium Epimerized N-sulfate K5 (EK5NS) by Diisopropylcarbodiimide (DIC), and both Sulfuric Acid and Hydrochloric Acid

Step 1: Conversion of EK5NS to a Pyridinium Salt. Same for Examples 17-A, 17-B, and 17-C as Step 1 in Example 16

### Example 17-A

Step 2: Induction of N→O Sulfate Transfer in EK5NS by Diisopropylcarbodiimide (DIC), and both Sulfuric Acid and Hydrochloric Acid

One hundred and fifteen milligrams of Py-EK5NS were dissolved in 6 milliliters of DMF. The solution was cooled to about 2° C. to about 4° C. in an ice-water bath. One hundred and six microliters of concentrated sulfuric acid was added to the solution with stirring. DIC (245 microliters) was added to this mixture. The reaction was stirred for 2 hours at about 2° C. to about 4° C. in an ice-water bath. Then, two hundred microliters of 4M HCl in dioxane was added to the solution with stirring. DIC (95 microliters) was added to this mixture. The reaction was carried out at about 2° C. to about 4° C. in an ice-water bath for additional 4 hours with stirring. At the end of the reaction period, the reaction solution was added to a mixture of 15 ml of dichloromethane (DCM) and 5 ml of 1N aqueous sodium hydroxide. After thorough mixing, the aqueous phase was separated from the organic phase by centrifugation at 1000 rpm in a Thermo Centra CL-2 centrifuge. The aqueous phase was removed from the centrifuge tube and kept at ambient temperature for about 30 minutes. The pH of the aqueous solution was then adjusted to about pH 6. The modified EK5NS formed in the N→O sulfate transfer reaction was isolated by ultrafiltration on a Millipore Centriprep apparatus with a sequence of 6 changes of water following the manufacturer's protocol. After ultrafiltration, the final volume was 5 ml.

### Example 17-B

Step 2: Induction of N→O Sulfate Transfer in EK5NS by Diisopropylcarbodiimide (DIC), and both Sulfuric Acid and Hydrochloric Acid

One hundred and fifteen milligrams of Py-EK5NS were dissolved in 6 milliliters of DMF. The solution was cooled to about 2° C. to about 4° C. in an ice-water bath. One hundred and six microliters of concentrated sulfuric acid was added to the solution with stirring. DIC (245 microliters) was added to this mixture. The reaction was stirred for 2 hours at about 2° C. to about 4° C. in an ice-water bath. Then, two hundred microliters of 4M HCl in dioxane was added to the solution with stirring. DIC (95 microliters) was added to this mixture. The reaction was carried out at about 2° C. to about 4° C. in an ice-water bath for additional 6 hours with stirring. At the end of the reaction period, the reaction solution was added to a mixture of 15 ml of dichloromethane (DCM) and 5 ml of 1N aqueous sodium hydroxide. After thorough mixing, the aqueous phase was separated from the organic phase by centrifugation at 1000 rpm in a Thermo Centra CL-2 centrifuge. The aqueous phase was removed from the centrifuge tube and kept at ambient temperature for about 30 minutes. The pH of the aqueous solution was then adjusted to about pH 6. The modified EK5NS formed in the N→O sulfate transfer reaction was isolated by ultrafiltration on a Millipore Centriprep apparatus with a sequence of 6 changes of water following the manufacturer's protocol. After ultrafiltration, the final volume was 5 ml.

### Example 17-C

Step 2: Induction of N→O Sulfate Transfer in EK5NS by Diisopropylcarbodiimide (DIC), and both Sulfuric Acid and Hydrochloric Acid

One hundred and fifteen milligrams of Py-EK5NS were dissolved in 6 milliliters of DMF. The solution was cooled to about 2° C. to about 4° C. in an ice-water bath. One hundred and six microliters of concentrated sulfuric acid was added to the solution with stirring. DIC (245 microliters) was added to this mixture. The reaction was stirred for 2 hours at about 2° C. to about 4° C. in an ice-water bath. Then, two hundred microliters of 4M HCl in dioxane was added to the solution with stirring. DIC (95 microliters) was added to this mixture. The reaction was carried out at about 2° C. to about 4° C. in an ice-water bath for additional 18 hours with stirring. At the end of the reaction period, the reaction solution was added to a mixture of 15 ml of dichloromethane (DCM) and 5 ml of 1N aqueous sodium hydroxide. After thorough mixing, the aqueous phase was separated from the organic phase by centrifugation at 1000 rpm in a Thermo Centra CL-2 centrifuge. The aqueous phase was removed from the centrifuge tube and kept at ambient temperature for about 30 minutes. The pH of the aqueous solution was then adjusted to about pH 6. The modified EK5NS formed in the N→O sulfate transfer reaction was isolated by ultrafiltration on a Millipore Centriprep apparatus with a sequence of 6 changes of water following the manufacturer's protocol. After ultrafiltration, the final volume was 5 ml.

Step 3: Examples 17-A, 17-B, and 17-C were re-N-sulfated as in Step 3 in Example 1-A

### Example 18

### O-sulfation by Pyridine Sulfur-trioxide Complex and Induction of N→O Sulfate Transfer in Pyridinium Epimerized N-sulfate K5 (EK5NS) by Diisopropylcarbodiimide (DIC) and Hydrochloric Acid in the Same Reaction Mixture

Step 1: Conversion of EK5NS to a Pyridinium Salt. Same for Examples 18-A and 18 B as Step 1 in Example 16

### Example 18-A

Step 2: O-sulfation by Pyridine Sulfur-trioxide Complex and Induction of N→O Sulfate Transfer in EK5NS by Diisopropylcarbodiimide (DIC) and Hydrochloric Acid in the Same Reaction Mixture.

One hundred and fifteen milligrams of Py-EK5NS were dissolved in 6 milliliters of DMF. The solution was cooled to about 2° C. to about 4° C. in an ice-water bath. One hundred and ninety milligrams of pyridine sulfur-trioxide complex was added to the solution with stirring. The reaction was stirred for 2 hours at about 2° C. to about 4° C. in an ice-water bath. Then, one hundred and fifty microliters of 4M HCl in dioxane was added to the solution with stirring. DIC (62 microliters) was added to this mixture. The reaction was carried out at about 2° C. to about 4° C. in an ice-water bath for additional 3 hours with stirring. At the end of the reaction period, the reaction solution was added to a mixture of 15 ml of dichloromethane (DCM) and 5 ml of 1N aqueous sodium hydroxide. After thorough mixing, the aqueous phase was separated from the organic phase by centrifugation at 1000 rpm in a Thermo Centra CL-2 centrifuge. The aqueous phase was removed from the centrifuge tube and kept at ambient temperature for about 30 minutes. The pH of the aqueous solution was then adjusted to about pH 6. The modified EK5NS formed in the N→O sulfate transfer reaction was isolated by ultrafiltration on a Millipore Centriprep apparatus with a sequence of 6 changes of water following the manufacturer's protocol. After ultrafiltration, the final volume was 5 ml.

### Example 18-B

Step 2: O-sulfation by Pyridine Sulfur-trioxide Complex and Induction of N→O Sulfate Transfer in EK5NS by Diisopropylcarbodiimide (DIC) and Hydrochloric Acid in the Same Reaction Mixture.

One hundred and fifteen milligrams of Py-EK5NS were dissolved in 6 milliliters of DMF. The solution was cooled to about 2° C. to about 4° C. in an ice-water bath. Three hundred and eighty milligrams of pyridine sulfur-trioxide complex was added to the solution with stirring. The reaction was stirred for 2 hours at about 2° C. to about 4° C. in an ice-water bath. Then, one hundred and fifty microliters of 4M HCl in dioxane was added to the solution with stirring. DIC (62 microliters) was added to this mixture. The reaction was carried out at about 2° C. to about 4° C. in an ice-water bath for additional 4 hours with stirring. At the end of the reaction period, the reaction solution was added to a mixture of 15 ml of dichloromethane (DCM) and 5 ml of 1N aqueous sodium hydroxide. After thorough mixing, the aqueous phase was separated from the organic phase by centrifugation at 1000 rpm in a Thermo Centra CL-2 centrifuge. The aqueous phase was removed from the centrifuge tube and kept at ambient temperature for about 30 minutes. The pH of the aqueous solution was then adjusted to about pH 6. The modified EK5NS formed in the N→O sulfate transfer reaction was isolated by ultrafiltration on a Millipore Centriprep apparatus with a sequence of 6 changes of water following the manufacturer's protocol. After ultrafiltration, the final volume was 5 ml.

Step 3: Examples 18-A and 18-B were re-N-sulfated as in Step 3 in Example 1-A

### Example 19

### Induction of N→O Sulfate Transfer in Pyridinium De-Acetylated, N-sulfated K5 (K5NS) by Diisopropylcarbodiimide (DIC) and Sulfuric Acid

Step 1: Conversion of K5NS to a Pyridinium Salt (Same for Examples 19-A, 16-B)

K5NS (0.5 gram) was dissolved in 30 milliliters of distilled water. A Dowex 50W-X8 ion exchange column (20-50 mesh, H⁺ form) was equilibrated with distilled water at a temperature range of about 2° C. to about 4° C. The K5NS solution was washed through the Dowex column with distilled water and the eluent was collected in a beaker. The pH of the solution was adjusted to a pH of 6.0 to 6.5 by addition of pyridine. The solution was lyophilized to obtain 0.45 gram of pyridinium-K5NS (Py-K5NS).

### Example 19-A

Step 2: Induction of N→O Sulfate Transfer in Py-K5NS by Diisopropylcarbodiimide (DIC) and Sulfuric Acid

One hundred and fifteen milligrams of Py-K5NS were dissolved in 6 milliliters of DMF. The solution was cooled to about 2° C. to about 4° C. in an ice-water bath. One hundred and six microliters of concentrated sulfuric acid was added to the solution with stirring. DIC (245 microliters) was added to this mixture. The reaction was stirred for 6 hours at about 2° C. to about 4° C. in an ice-water bath. At the end of the reaction period, the reaction solution was added to a mixture of 15 ml of dichloromethane (DCM) and 5 ml of 1N aqueous sodium hydroxide. After thorough mixing, the aqueous phase was separated from the organic phase by centrifugation at 1000 rpm in a Thermo Centra CL-2 centrifuge. The aqueous phase was removed from the centrifuge tube and kept at ambient temperature for about 30 minutes. The pH of the aqueous solution was then adjusted to about pH 6. The modified K5NS formed in the N→O sulfate transfer reaction was isolated by ultrafiltration on a Millipore Centriprep apparatus with a sequence of 6 changes of water following the manufacturer's protocol. After ultrafiltration, the final volume was 5 ml. The solution was lyophilized to dryness.

H1 NMR analysis of the product showed that at least about 30% of glucosamine residues of the K5NS contain both 3-O-sulfate and N-sulfate.

### Example 19-A1

The anticoagulant activity was tested for anti-Xa activity according the method in Andersson et al. Thrombosis Research 9 575 (1976) using USP heparin as standard. The anti-Xa activity of Example 19-A was 120 upper milligram Example 19-B

Step 2: Induction of N→O Sulfate Transfer in K5NS by Diisopropylcarbodiimide (DIC) and Sulfuric Acid

One hundred and fifteen milligrams of Py-K5NS were dissolved in 6 milliliters of DMF. The solution was cooled to about 2° C. to about 4° C. in an ice-water bath. One hundred and six microliters of concentrated sulfuric acid was added to the solution with stirring. DIC (245 microliters) was added to this mixture. The reaction was stirred for 16 hours at about 2° C. to about 4° C. in an ice-water bath. At the end of the reaction period, the reaction solution was added to a mixture of 15 ml of dichloromethane (DCM) and 5 ml of 1N aqueous sodium hydroxide. After thorough mixing, the aqueous phase was separated from the organic phase by centrifugation at 1000 rpm in a Thermo Centra CL-2 centrifuge. The aqueous phase was removed from the centrifuge tube and kept at ambient temperature for about 30 minutes. The pH of the aqueous solution was then adjusted to about pH 6. The modified K5NS formed in the N→O sulfate transfer reaction was isolated by ultrafiltration on a Millipore Centriprep apparatus with a sequence of 6 changes of water following the manufacturer's protocol. After ultrafiltration, the final volume was 5 ml. The solution was lyophilized to dryness.

H1 NMR analysis of the product showed that at least about 60% of glucosamine residues of the K5NS contain both 3-O-sulfate and N-sulfate.

### Example 19-B1

The anticoagulant activity was tested for anti-Xa activity according the method in Andersson et al. Thrombosis Research 9 575 (1976) using USP heparin as standard. The anti-Xa activity of Example 19-B was 150 upper milligram. The anti-IIa activity of Example 19-B was 225 upper milligram

### Example 20

### Induction of N→O Sulfate Transfer in Pyridinium Heparin by Diisopropylcarbodiimide (DIC) and Hydrochloric acid

Step 1: Same as Step 1 in Example 1-A

Step 2: Induction of N→O Sulfate Transfer in Pyridinium Heparin by Diisopropylcarbodiimide (DIC) and Hydrochloric Acid

One hundred and fifty milligrams of Py-heparin were dissolved in 6 milliliters of DMF. The solution was cooled to about 2° C. to about 4° C. in an ice water bath. Eight hundred microliters of 4M HCl in dioxane was added to the solution with stirring. DIC (185 microliters) was added to this mixture. The reaction was carried out at about 2° C. to about 4° C. in an ice-water bath for 4 hours with stirring. At the end of the reaction period, the reaction mixture was added to a mixture of 15 ml of dichloromethane (DCM) and 5 ml of 1N sodium hydroxide. After thoroughly mixing, the aqueous phase was separated from organic phase by centrifugation at 1000 rpm in a Thermo Centra CL-2 centrifuge. The aqueous phase was removed from the centrifuge tube and kept at ambient temperature for about 30 minutes. The pH of the aqueous solution was then adjusted to about pH 6. The structurally modified heparin formed in the N→O sulfate transfer reaction was isolated by ultrafiltration using Millipore Centriprep apparatus a sequence of 6 changes of water following the manufacturer's protocol. After ultrafiltration, the final volume is 5 ml.

Step 3: Re—N-sulfation of the Modified Heparin from Step 2. Same as Step 3 in Example 1-A

Analytical Result:

H1 NMR analysis of the product showed that at least about 30% of glucosamine residues of the heparin contain both 3-O-sulfate and N-sulfate.

Anticoagulation Activities:

Example 20 was tested same as Example 19-B1. The anti-Xa activity was 180 upper milligram.

The various compositions of the present invention will preferably be used alone or in combination with pharmaceutically acceptable excipient materials to treat certain diseases. Preferred pharmacologically acceptable excipients include neutral saline solutions buffered with phosphate, lactate, Tris, and other appropriate buffers known in the art.

Examples of the types of disease that can be treated with compositions of the invention include cardiovascular disease, and cancer. In the former instance, the compositions have preferable applications for the treatment of arterial thrombosis, venous thrombosis, atherosclerosis, prevention of arterial stenosis and re-stenosis following mechanical interventions. As applied to cancer, the compositions can prevent the spread, or metatasis, of cancer.

What is claimed is:

1. A process for structural modification of a substrate molecule comprising an oxyanion in an amide or ester linkage and a hydroxyl group wherein the oxyanion is caused to undergo regioselective intramolecular migration to the hydroxyl group, said process comprising:
a) converting said substrate molecule into an amine salt;
b) dissolving the amine salt of said substrate molecule in an aprotic solvent to form a solution; and
c) treating said solution with an activating agent to cause said oxyanion to regioselectively migrate to said hydroxyl group.

2. The process according to claim 1, wherein in step (c) the solution is treated with an acidic catalyst and the activating agent.

3. The process according to claim 2, wherein the activating agent comprising an agent that activates the oxyanion to cause a migration of the oxyanion to the hydrosyl group

4. The process according to claim 2, wherein the activating agent is selected from the group consisting of of dicyclohexylcarbodiimide, diisopropylcarbodiimide, 3-ethyl-1-(3-dimethylaminopropyl)carbodiimide hydrochloride, 1-cyclohexyl-3-(2-morpholoethyl)carbodiimide p-toluene, 1-hydroxybenzotriazole, N-hydroxysuccinimide, Benzotriazol-1-yloxytris(dimethylamine) phosphonium hexafluorophosphate (BOP), o-benzotriazol-1-yl-N,N,N′,N′-tetramethyluronium hexafluorophosphate (HBTU), Bis(2-oxo-3-oxazolidinyl)phosphinic chloride (BOP-Cl), (1H-1,2,3-benzotriazol-1-yloxy)tris(pyrrolidino)phosphonium hexafluorophosphate (Py-BOP), carbonyldiimidazole, and 2-chloro-1-methylpyridinium Iodide.

5. The process according to claim 2, wherein the acid catalyst is selected from the group consisting of sulfuric acid, phosphoric acid, hydrochloric acid, hydrofluoric acid, hydrobromide acid, and hydroiodic acid.

6. The process according to claim 2, wherein the concentration of acid catalyst is between approximately 0.1 molar and 100 molar.

7. The process according to claim 2, wherein step (c) is carried out at a temperature from about minus 20 degrees Centigrade to about 60 degrees Centigrade.

8. The process according to claim 2, wherein step (c) is carried out for a period from about 0.5 hours to about 48 hours.

9. The process according to claim 1, wherein the substrate molecule is heparin, or oligosaccharides derived from heparin.

10. The process according to claim 2, wherein the substrate molecule is heparin, or oligosaccharides derived from heparin.

11. The process according to claim 1, wherein the substrate molecule is heparan sulfate, or oligosaccharides derived from heparan sulfate.

12. The process according to claim 1, wherein the substrate molecule is N-deacetylated, N-sulfated heparin, or oligosaccharides derived from N-deacetylated, N-sulfated heparin.

13. The process according to claim 1, wherein the substrate molecule is N-deacetylated, N-sulfated heparan sulfate, or oligosaccharides derived from N-deacetylated, N-sulfated heparan sulfate.

14. The process according to claim 1, wherein the substrate molecule is N-deacetylated, N-sulfated chondroitin sulfate, or oligosaccharides derived from N-deacetylated, N-sulfated chondroitin sulfate.

15. The process according to claim 1, wherein the substrate molecule is N-deacetylated, N-sulfated dermatan sulfate, or oligosaccharides derived from N-deacetylated, N-sulfated dermatan sulfate.

16. The process according to claim 1, wherein the substrate molecule is N-deacetylated, N-sulfated hyaluronic acid, or oligosaccharides derived from N-deacetylated, N-sulfated hyaluronic acid.

17. The process according to claim 1, wherein the substrate molecule is N-deacetylated, N-sulfated chitin, or oligosaccharides derived from N-deacetylated, N-sulfated chitin.

18. The process according to claim 1, wherein the substrate molecule is N-deacetylated, N-sulfated polysaccharides containing N-acetylated glucosamine, galactosamine, or mannosamine, or oligosaccharides derived from N-deacetylated, N-sulfated said polysaccharides.

19. The process according to claim 18, wherein said polysaccharides containing N-acetylated amino sugars are selected from the group consisting of the E. coli polysaccharides K5, the E. coli polysaccharide K4, the polysaccharides acharan sulfate, and the polysaccharides derived from bacteria, fungi, plant and animals.

20. The process according to claim 1, wherein the oxyanion on said substrate molecule undergoes intramolecular transfer to a hydroxyl group that is adjacent to, or nearby, the original oxyanion amide or ester and, simultaneously, said substrate molecule is O-sulfated at one or more hydroxyl groups that are not adjacent to, or nearby, the original oxyanion amide or ester, said process comprising:
a) converting said substrate molecule into an amine salt soluble in an aprotic solvent;
b) dissolving the amine salt of said substrate in an aprotic solvent to form a first solution; and
c) treating said first solution with an N,N′-carbodiimide and a sulfuric acid catalyst under conditions of temperature, time, and solvent optimized to give maximal rates and highest regioselectivity in the oxyanion transfer reaction as well as O-sulfation.

21. The process according to claim 20, wherein said amine salt in step (a) is either a quaternary amine salt or a tertiary amine salt.

22. The process according to claim 21, wherein said tertiary amine salt is a member selected from the group consisting of trimethylamine, triethylamine, tripropylamine, tributylamine, and pyridine.

23. The process according to claim 21, wherein said quaternary amine salt is a member selected from the group consisting of cetylpyridium and cetyltrimethylammonium salts.

24. The process according to claim 20, wherein the solvent in step (b) is a member selected from a group consisting of dimethylformamide, dimethylsulfoxide, chloromethane, chloroform, and terahydrofurane.

25. The process according to claim 20, wherein said N,N′-carbodiimide used in step (c) is a member selected from the group consisting of dicyclohexylcarbodiimide, diisopropylcarbodiimide, 3-ethyl-1-(3-dimethylaminopropyl)carbodiimide hydrochloride, and 1-cyclohexyl-3-(2-morpholoethyl)carbodiimide p-toluene, 1-hydroxybenzotriazole, N-hydroxysuccinimide, Benzotriazol-1-yloxytris(dimethylamine) phosphonium hexafluorophosphate (BOP), o-benzotriazol-1-yl-N,N,N′,N′-tetramethyluronium hexafluorophosphate (HBTU), Bis(2-oxo-3-oxazolidinyl)phosphinic chloride (BOP-Cl), (1H-1,2,3-benzotriazol-1-yloxy)tris(pyrrolidino)phosphonium hexafluorophosphate (Py-BOP), carbonyldiimidazole, and 2-chloro-1-methylpyridinium Iodide.

26. The process according to claim 20, wherein step (c) is carried out at a temperature from about 2 degrees Centigrade to about 60 degrees Centigrade.

27. The process according to claim 20, wherein the reaction in step (c) is carried out for a period from about 2 hours to about 24 hours.

28. The process according to claim 20, wherein the substrate molecule is heparin or oligosaccharides derived from heparin.

29. The process according to claim 20, wherein the substrate molecule is heparan sulfate or oligosaccharides derived from heparan sulfate.

30. The process according to claim 20, wherein the substrate molecule is N-deacetylated, N-sulfated heparin or oligosaccharides derived from N-deacetylated, N-sulfated heparin.

31. The process according to claim 20, wherein the substrate molecule is N-deacetylated, N-sulfated heparan sulfate or oligosaccharides derived from N-deacetylated, N-sulfated heparan sulfate.

32. The process according to claim 20, wherein the substrate molecule is N-deacetylated, N-sulfated chondroitin sulfate or oligosaccharides derived from N-deacetylated, N-sulfated chondroitin sulfate.

33. The process according to claim 20, wherein the substrate molecule is N-deacetylated, N-sulfated dermatan sulfate or oligosaccharides derived from N-deacetylated, N-sulfated dermatan sulfate.

34. The process according to claim 20, wherein the substrate molecule is N-deacetylated, N-sulfated hyaluronic acid or oligosaccharides derived from N-deacetylated, N-sulfated hyaluronic acid.

35. The process according to claim 20, wherein the substrate molecule is N-deacetylated, N-sulfated chitin or oligosaccharides derived from N-deacetylated, N-sulfated chitin.

36. The process according to claim 20, wherein the substrate molecule is N-deacetylated, N-sulfated polysaccharides containing N-acetylated glucosamine, galactosamine, or mannosamine, or oligosaccharides derived from N-deacetylated, N-sulfated polysaccharides.

37. The process according to claim 36, wherein said polysaccharides containing N-acetylated amino sugars comprise the polysaccharides derived from E. coli strains K4 or K5, the polysaccharides acharan sulfate, or the polysaccharides derived from bacteria, fungi, plant and animals.

38. A composition, of a resulting molecule made according to claim 1.

39. A composition of derivatives of heparin, heparan sulfate, chondroitin sulfate, dermatan sulfate, hyaluronic acid, polysaccharide K5, polysaccharide K4 and other polysaccharides, their derivatives, their derived oligosaccharides, and their derived monosaccharides, made according to claim 1.

40. A composition of polysaccharides, oligosaccharide and monosaccharide containing an amino sugar, which amino sugar contains the hydroxyloxyanion, such as sulfate and phosphate, in the range from 0.01 moles to 1.00 moles of hydroxyl oxianion per amino sugar.

41. A composition of heparin and heparin derived oligosaccharides that comprise newly formed sulfate esters on glucosamine or uronic acid residues, in the range from 0.01 moles to 1.00 moles of sulfate ester per mole of glucosamine or uronic acis residues, wherein sulfate esters are caused by intramolecular migration from N-sulfate of glucosamine, according to claim 20.

42. A composition according to claim 41 utilitzed as a therapeutic or pharmaceutical agent to treat diseases comprising thrombosis, atherosclerosis, metastasis, angiogenesis, and inflammatory diseases.

43. A method of treating or preventing disease, comprising the steps of administering a sufficient amount of a composition of matter as described in claim 42 for a time sufficient to treat or prevent said disease, and repeating said administration if desired.

44. A method of treating or preventing disease as described in claim 43, wherein said disease is a cardiovascular disease.

45. A method of treating or preventing disease as described in claim 43, wherein said disease is cancer.
